(19) United States
(12) Patent Application Publication
(10) Pub. No.: US 2017/0007690 A1
(43) Pub. Date: Jan. 12, 2017

(54) METHODS AND COMPOSITIONS FOR THE STABILIZATION OF ANTHRAX RECOMBINANT PROTECTIVE ANTIGEN

(71) Applicant: NanoBio Corporation, Ann Arbor, MI (US)

(72) Inventor: Susan Ciotti, Ann Arbor, MI (US)

(73) Assignee: NanoBio Corporation, Ann Arbor, MI (US)

(21) Appl. No.: 15/260,145

(22) Filed: Sep. 8, 2016

Related U.S. Application Data
(63) Continuation-in-part of application No. 15/203,387, filed on Jul. 6, 2016.
(60) Provisional application No. 62/218,363, filed on Sep. 14, 2015.
(60) Provisional application No. 62/189,595, filed on Jul. 7, 2015.
(60) Provisional application No. 62/218,320, filed on Sep. 14, 2015.

(51) Int. Cl. A61K 39/07; A61K 9/08; A61K 47/26; A61K 47/18; A61K 47/02

(52) U.S. Cl. CPC A61K 39/07; A61K 47/18; A61K 47/02; A61K 47/26; A61K 47/183; A61K 9/08

(57) ABSTRACT

The present disclosure relates to buffer-stabilized anthrax recombinant protective antigen (rPA) compositions and methods of making the same. The disclosed compositions and methods provide a means of stabilizing and preserving rPA in such a way that the protein maintains its native conformation and structure, maintains its immunological activity, and prevents aggregation.

### CROSS-REFERENCE TO RELATED APPLICATIONS

This application claims priority from U.S. Provisional Patent Application No. 62/218,363, filed Sep. 14, 2015. This application is also a Continuation-in-Part of U.S. patent application Ser. No. 15/203,387, filed Jul. 6, 2016, which claims priority from U.S. Patent Application No. 62/218,320, filed Sep. 14, 2015, and 62/189,595, filed Jul. 7, 2015. The contents of these applications are incorporated herein by reference in their entirety.

### FIELD OF THE APPLICATION

The present application is directed to methods and compositions for stabilizing recombinant protective antigen (rPA) of anthrax. The disclosed compositions and methods of stabilization may be useful in the formulation, storage, and transportation of a variety of pharmaceutical, therapeutic, and/or research compositions comprising recombinant protective antigen (rPA) of anthrax.

### BACKGROUND OF THE INVENTION

A. Protein Stabilization

To stabilize labile products, some try to immobilize or reduce the water content of stored samples. For example, some biological materials can be stabilized by chilling or freezing. However, maintaining and transporting frozen samples is costly, and freezer breakdown may result in the complete loss of valuable product. Alternatively, bio-products can be freeze-dried to provide a dry, active, shelf-stable, and readily soluble product. However, a protein or biologic drug product can be damaged during the freeze-drying process in numerous ways. Often regarded as a gentle method, freeze drying is in reality a potentially damaging process where the individual process stages should be regarded as a series of interrelated stresses, each of which can damage sensitive bio-products. Damage sustained during one step in the process may be exacerbated at succeeding stages in the process chain, and even apparently trivial changes in the process, such as a change in container, may be sufficient to transform a successful process to one which is unacceptable. Reducing temperature in the presence of ice formation is the first major stress imposed on a biomolecule. Biomolecules in vaccine products are more likely to be damaged by an increase in solute concentration as ice forms. Further, freeze-drying is less appropriate for oily or non-aqueous solutions where the material has a low melting temperature.

B. Proteins in Vaccines

Immunization is a principal feature for improving the health of people. Despite the availability of a variety of successful vaccines against many common illnesses, infectious diseases remain a leading cause of health problems and death. Significant problems inherent in existing vaccines include the need for repeated immunizations, and the ineffectiveness of the current vaccine delivery systems for a broad spectrum of diseases.

One problem present in the art is the frequent denaturation of protein antigens present in vaccine formulations. Many vaccines comprise protein antigens to confer protective immunity. This is because antibodies are most likely to be protective if they bind to the surface of the invading pathogen triggering its destruction. Several vaccines employ purified surface molecules. For example, influenza vaccine contains purified hemagglutinins from the viruses currently in circulation around the world. In addition, the gene encoding a protein expressed on the surface of the hepatitis B virus, called hepatitis B surface antigen or HBsAg, can now be expressed in E. coli cells and provides the material for an effective vaccine. The genes encoding the capsid proteins of 4 strains of human papilloma virus (HPV) can be expressed in yeast and the resulting recombinant proteins are incorporated in a vaccine (Gardasil®). Because infection with some of these strains of HPV can lead to cervical cancer, the HPV vaccine is useful to prevent certain types of cancer.

Other types of vaccines can utilize a poor (polysaccharide organism) antigen coupled to a carrier protein (preferably from the same microorganism), thereby conferring the immunological attributes of the carrier on the attached antigen. This technique for the creation of an effective immunogen is most often applied to bacterial polysaccharides for the prevention of invasive bacterial disease.

One disadvantage of vaccines comprising protein antigens or a carrier protein is that the protein present in the vaccine formulation can become unstable, resulting in protein denaturation. Denaturation of a protein antigen can produce loss in effective binding, and thereby a decrease in production of protective antibodies. Similarly, denaturation of a carrier protein present in a conjugate vaccine can also result in loss in effective binding, and thereby a decrease in production of protective antibodies.

Thus, it would be a great advance in the field if vaccine products comprising a protein antigen or a carrier protein could be stabilized without the need for freeze-drying or storage conditions at below sub-zero temperatures (−20 to −80° C.). Developing a stabile liquid-based solution that extends the shelf-life of a protein present in a vaccine composition at simple refrigerated temperatures (2 to 8° C.) or, more importantly, room temperature (25° C.) would greatly reduce the manufacturing costs (e.g. freeze-drying cost prohibitive) and supply chain needs for products that need storage at −20° C. to −80° C.

There remains a need in the art for effective stabilization and preservation of recombinant protective antigen (rPA) of anthrax for all kinds of pharmaceutical, therapeutic, and research indications. The present disclosure satisfies these needs.

### SUMMARY OF INVENTION

The present disclosure relates primarily to methods and compositions of stabilizing and preserving recombinant protective antigen (rPA) in solution. The disclosed methods and compositions will be useful for research as well as therapeutic purposes.

In one aspect, the present disclosure relates to a method of stabilizing anthrax recombinant protective antigen (rPA) in a composition, comprising formulating the protein in a stabilizing system. The stabilizing system comprises: (a) tris(hydroxymethyl)aminomethane (TRIS) buffer; (b) at least one salt; (c) at least one sugar; and (d) at least one amino acid. In some embodiments, the TRIS buffer is present in a concentration of about 5-about 100 mM. In some embodiments, the TRIS buffer is present in a concentration of about 10 mM or about about 80 mM.

In some embodiments, the salt is sodium chloride, while in other embodiments, the salt is calcium chloride. In some embodiments, the concentration of the salt is about 50-about 150 mM.

In some embodiments, the sugar is trehalose, and in some embodiments, the concentration of trehalose is about 5-about 15%. In some embodiments, the amino acid is histidine. In some embodiments, the histidine is present in a concentration of about 20-about 70 mM, or, more specifically, about 60 mM.

The disclosure encompasses a composition comprising anthrax recombinant protective antigen (rPA) in a stabilizing system, wherein the stabilizing system comprises: (a) TRIS buffer; (b) at least one salt; (c) at least one sugar; and (d) at least one amino acid. In some embodiments, the TRIS buffer is present in a concentration of about 5-about 100 mM. In some embodiments, the TRIS buffer is present in a concentration of about 10 mM or about 80 mM.

In some embodiments, the salt present in the composition is sodium chloride, while in other embodiments, the salt is calcium chloride. In some embodiments, the concentration of the salt is about 50-about 150 mM.

In some embodiments, the sugar present in the composition is trehalose, and in some embodiments, the concentration of trehalose is about 5-about 15%. In some embodiments, the amino acid is histidine. In some embodiments, the histidine is present in a concentration of about 20-70 mM, or, more specifically, about 60 mM.

In some embodiments, the composition can be formulated into a pharmaceutical composition, for instance, a vaccine.

The foregoing general description and following brief description of the drawings and the detailed description are exemplary and explanatory and are intended to provide further explanation of the disclosed as claimed. Other objects, advantages, and novel features will be readily apparent to those skilled in the art from the following detailed description of the disclosed.

### BRIEF DESCRIPTION OF DRAWINGS

FIG. 1 shows an example of protein denaturation.

FIG. 2 shows a flowchart of Prototype 1.

FIG. 3 shows a flowchart of Prototype 2.

FIG. 4 shows a flowchart of Prototype 3.

FIG. 5 shows the best pH for protection against aggregation of anthrax protective antigen (rPA).

FIG. 6 shows the effect of the concentration of trehelose on the stabilization of rPA (showing six different concentrations of trehalose).

FIG. 7 shows crystallization of mannitol in buffer store at 2-8° C. for four weeks.

FIG. 8 shows a ribbon diagram of the tertiary structure of rPA showing the domains: d1, d2, d3, and d4, and where * indicates calcium atoms are binding.

FIG. 9 shows SEC-HPLC chromatograph of rPA solution after incubation at 49° C. for 1 and 5 minutes

FIG. 10 shows the effect of temperature and time on rPA physical stability using PAGE gels.

FIG. 11 shows the physical appearance of 500 μg/ml rPA in sodium phosphate systems with different excipients: Non-heated Control (left vial), Heating at 49° C. for 5 minutes (right vial).

FIG. 12 shows comparisons of rPA peak area as determined by SEC-HPLC of rPA in phosphate buffered solutions (PBS) with additional stabilizing excipients. Panel (A) shows formulations without histidine and panel (B) shows formulations with histidine.

FIG. 13 shows the physical appearance of 500 μg/ml rPA in TRIS buffer with different excipients following heating at 49° C. for 5 Minutes

FIG. 14 shows a comparison of rPA Peak area as determined by SEC-HPLC of various with TRIS Buffer Formulations. Some peaks include rPA+20% W805EC nanoemulsion.

FIG. 15 shows SEC-HPLC chromatographs of rPA in various excipients.

FIG. 16 shows examples of physical acceptance criteria of rPA buffered aqueous solutions.

FIG. 17 shows examples of physical acceptance criteria of rPA buffered aqueous solutions.

FIG. 18 shows the particle size profile of 100 μg/mL rPA aqueous solution (Prototype 1: X-1596). Panel (A) shows stability data at 1 month at −20° C., 5° C., and 25° C., and panel (B) shows stability data at 1 month at 5° C. and 40° C.

FIG. 19 shows rPA aqueous (AQ) (5% Trehalose) formulations by temperature and month.

FIG. 20 shows rPA aqueous (AQ) (15% Trehalose) formulations by temperature and month.

FIG. 21 shows rPA aqueous (AQ) (P3−GT) formulations by temperature and month.

FIG. 22 shows rPA aqueous (AQ) (P3+GT) formulations by temperature and month.

FIG. 23 shows rPA Aqueous solution stability of low dose rPA over 12 months.

Panels (A) and (B) show formulations without glutathione and panels (C) and (D) show formulations with glutathione.

FIG. 24 shows rPA aqueous solution stability of high dose rPA aqueous solutions. 12 months of rPA stability was measured after storage at −20 C, 5 C, 25° C. (RP/SEC, +GT). Panels (A) and (B) show formulations without glutathione and panels (C) and (D) show formulations with glutathione.

FIG. 25 shows a ribbon diagram of the tertiary structure of rPA showing the nine peptide epitopes that exhibited significantly and reproducibly stronger reactivity to sera from mice immunized with rPA-Alhydrogel formulations stored for 3 weeks at either room temperature (RT) or 37° C. than to sera from mice immunized with freshly prepared formulations. Peptides are numbered with the residue number of the first amino acid of the 12-mer peptide sequence.

FIG. 26 shows pH assessment of Prototype 1 rPA formulations over time. (A) and (B) show formulations with 100 μg of rPA and panels (C) and (D) show formulations with 500 μg of rPA.

FIG. 27 shows pH assessment of Prototype 2 rPA formulations over time. (A) and (B) show formulations with 100 μg of rPA and panels (C) and (D) show formulations with 500 μg of rPA.

FIG. 28 shows pH assessment of Prototype 3 rPA formulations over time. (A) and (B) show formulations with 100 μg of rPA and panels (C) and (D) show formulations with 500 μg of rPA.

FIG. 29 shows the acceptance criteria for the qualitative Western Blot method.

### DETAILED DESCRIPTION

### I. Overview

The primary purpose of the disclosed compositions and methods is to achieve long-term stability, including preserved biological function and structure, of rPA in an aqueous formulation. It is known that stabilizing agents/excipients may be added to formulations to increase shelf-life of a product. However, the state of the art leaves much to be desired. The present disclosure utilized various novel screening methodologies to select excipients that provide additional thermo-labile protection for rPA present in an aqueous formulation.

Table 2 below describes some of the exemplary buffer systems and additional stabilizing excipients that were developed as part of the present disclosure. These systems were heat screened in stability studies, which may be used to guide formulation development and choose specific excipients.

A. Anthrax Protective Antigen for the Disclosed Methods and Compositions

Anthrax protective antigen, to which the present disclosure applies, may be generated by biosynthesis using recombinant DNA technology and are referred to herein as “recombinant proteins” or “recombinantly produced proteins.” The skilled reader will know how to use recombinant technology to biosynthesize the proteins and precursor proteins of the present disclosure.

The novel formulations of the present disclosure retain the physical, chemical, and biological stability of the rPA protein incorporated therein, and prevent the rPA protein, which may be intended for administration into a subject, from forming aggregates and/or particulates. The disclosed compositions and methods further prevent rPA protein denaturation and preserve the stabilized rPA protein in solution for an extended period of time.

The term “folded globular protein” refers to a protein in its properly folded, three-dimensional conformation, and includes the designed, desired, or required arrangement of disulfide bonds linking cysteine residues of a protein. Usually, this properly folded disulfide arrangement will be identical to or comparable to that present in its analogous native protein. Preferably, folded proteins stabilized by the process of the present disclosure will have two or more disulfide bonds. rPA is an example of a “folded globular protein.”

Globular proteins are more soluble in aqueous solutions, and are generally more sensitive to temperature and pH change than are their fibrous counterparts; furthermore, they do not have the high glycine content or the repetitious sequences of the fibrous proteins. Globular proteins incorporate a variety of amino acids, many with large side chains and reactive functional groups. The interactions of these substituents, both polar and nonpolar, often cause the protein to fold into spherical conformations which gives this class its name. In contrast to the structural function played by the fibrous proteins, the globular proteins are chemically reactive, serving as enzymes (catalysts), transport agents and regulatory messengers. Such proteins are generally more sensitive to temperature and pH change than their fibrous counterparts.

Heat is one factor that effects protein conformation and structure. The term thermolabile refers to a substance which is subject to destruction/decomposition or change in response to heat. This term is often used to describe biochemical substances, including proteins. A protein or peptide may lose activity due to changes in the three-dimensional structure of the protein during exposure to heat. Many proteins, including rPA, are thermolabile. Heat denaturation is primarily due to the increased entropic effects of the non-polar residues (that is, the increased entropy gain of the unfolded chain is not much reduced by the small amount of entropy loss caused to the solute).

rPA is a globular protein having a tertiary structure. Tertiary structures of globular proteins (“Folded Globular Proteins”) involves electrostatic interactions, hydrogen bonding and covalent disulfide bridges. These are areas with barrel shapes known as domains. Each domain is a region within the native tertiary structure that can potentially exist independent of the protein or antigenic peptide epitopes. These include hydrophobic attraction of nonpolar side chains in contact regions of the subunits, electrostatic interactions between ionic groups of opposite charge: hydrogen bonds between polar groups; and disulfide bonds.

B. Issues Related to Protein Structure Stabilization

There are four parts to protein stabilization: protein hydration, protein folding, protein crystallization, and protein denaturation.

Protein hydration: When a protein is fully hydrated, the potential energy is reduced and the proteins can attain their minimum-energy conformation. The water molecules can lubricate the movement of the amino acids backbone and the side groups for exchange of hydrogen bonds. Such water promotes both folding rate and stability of the protein.

Protein folding: Protein folding is driven by the aqueous environment, particularly the hydrophobic interactions, due to the unfavorable entropy decrease (mostly translational forming a large surface area of non-polar groups with water). Consider a water molecule next to a surface to which it cannot hydrogen bond. The incompatibility of this surface with the low-density water that forms over such a surface encourages the surface minimization that drives the proteins' tertiary structure formation. Compatible solutes or osmolytes can stabilize the surface low-density water and increase the surface tension, thus to stabilize the protein's structure (Hofmeister effect and the solubility of non-polar gases).

Protein crystallization: Proteins may form crystals when precipitated slowly from an aqueous solution (e.g. of ammonium sulfate). Slow precipitation is required to produce small numbers of larger crystals rather than very large numbers of small crystals. Crystals of un-denatured proteins for structural analysis are best formed with water molecules retained within the crystal lattice. Crystallization of native proteins appears to have a three-step mechanism involving nucleation, in which mesoscopic metastable protein clusters of dense liquid serve as precursors to the ordered crystal nuclei followed by crystal growth. This process seems to involve an aqueous biphasic separation and fits nicely with the two-state structuring in liquid water, where the crystallization takes place within the dense phase.

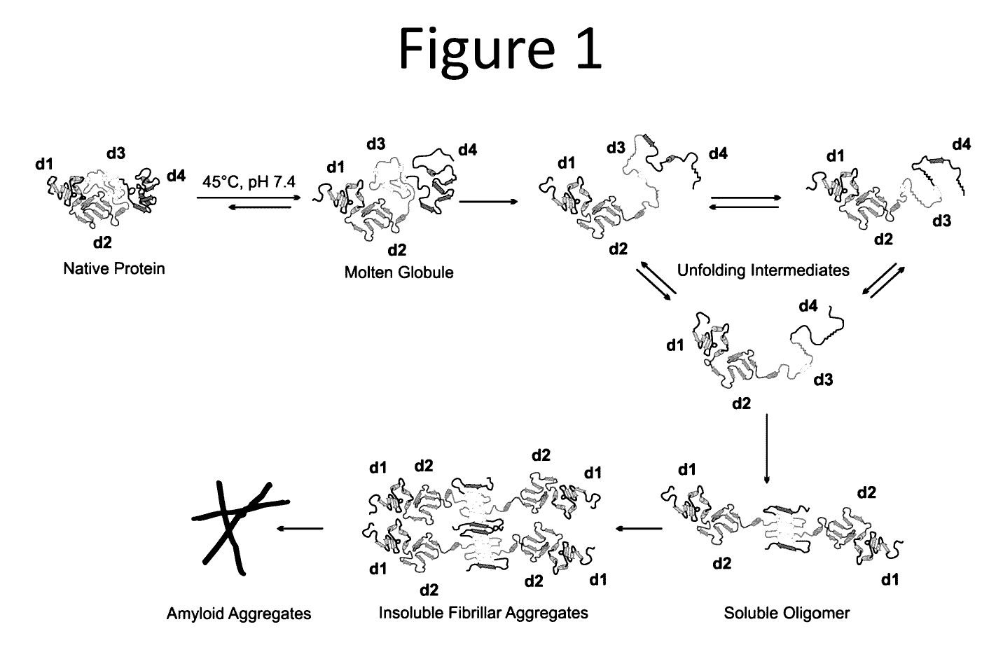

Protein denaturation: Protein denaturation involves a change in the protein structure (generally an unfolding) with the loss of activity, as shown in FIG. 1. Water is critical, not only for the correct folding of proteins but also for the maintenance of this structure. Heat denaturation and loss of biological activity has been linked to the breakup of the 2-D-spanning water network (see above) around the protein (due to increasing hydrogen bond breakage with temperature), which otherwise acts restrictively on protein vibrational dynamics. The free energy change on folding or unfolding is due to the combined effects of both protein folding/unfolding and hydration changes. These compensate to such a large extent that the free energy of stability of a typical protein is only 40-90 kJ mol⁻¹ (equivalent to very few hydrogen bonds), whereas the enthalpy change (and temperature times the entropy change) may be greater than ±500 kJ mol⁻¹ different. There are both enthalpic and entropic contributions to this free energy that change with temperature and so give rise to heat denaturation and, in some cases, cold denaturation. Protein unfolding at higher temperatures (heat denaturation) is easily understood but the widespread existence of protein unfolding at low temperatures is surprising, particularly as it is unexpectedly accompanied by a decrease in entropy.

The methods and compositions of the present disclosure address the issues of rPA protein stabilization by stabilizing the protein in solution such that rPA retains its structure, conformation, and immunological activity. The type of stabilization provided by the disclosure is valuable scientifically, academically, and commercially for the research, development, commercialization, and treatment/administration of rPA-based therapeutics, including vaccines.

For the purposed of the disclosed compositions and methods, rPA may be incorporated into disclosed stabilizing systems in varying amounts, as necessary. For instance, a formulation of the disclosed compositions and methods may contain a concentration of rPA in ranges between 1-5000 μg/ml, between 10-1000 μg/ml, between 50-750 μg/ml, or between 100-500 μg/ml. In other words, the concentration of rPA in the disclosed compositions and methods can be about about 1, about 2, about 3, about 4, about 5, about 6, about 7, about 8, about 9, about 10, about 20, about 30, about 40, about 50, about 60, about 70, about 80, about 90, about 100, about 125, about 150, about 175, about 200, about 225, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, about 1000, about 1050, about 1100, about 1150, about 1200, about 1250, about 1300, about 1350, about 1400, about 1450, about 1500, about 1550, about 1600, about 1650, about 1700, about 1750, about 1800, about 1850, about 1900, about 1950, about 2000, about 2050, about 2100, about 2150, about 2200, about 2250, about 2300, about 2350, about 2400, about 2450, about 2500, about 2550, about 2600, about 2650, about 2700, about 2750, about 2800, about 2850, about 2900, about 2950, about 3000, about 3050, about 3100, about 3150, about 3200, about 3250, about 3300, about 3350, about 3400, about 3450, about 3500, about 3550, about 3600, about 3650, about 3700, about 3750, about 3800, about 3850, about 3900, about 3950, about 4000, about 4050, about 4100, about 4150, about 4200, about 4250, about 4300, about 4350, about 4400, about 4450, about 4500, about 4550, about 4600, about 4650, about 4700, about 4750, about 4800, about 4850, about 4900, about 4950, or about 5000 μg/ml.

### II. Novel Methods to Stabilize rPA

The present disclosure is directed to methods of optimizing compositions to stabilize the secondary and tertiary structures of rPA, by proactively screening and addressing all the destabilizing or un-stabilizing factors that would affect the rPA protein structure and lead to aggregation and degradation of the rPA protein.

A. Carbohydrates or Sugars

Hydrophobic Effect: The major driving force in protein folding is the hydrophobic effect. This is the tendency for hydrophobic molecules to isolate themselves from contact with water. As a consequence during protein folding the hydrophobic side chains become buried in the interior of the protein. The exact physical explanation of the behavior of hydrophobic molecules in water is complex and can best be described in terms of their thermodynamic properties. Much of what is known about the hydrophobic effect has been derived from studying the transfer of hydrocarbons from the liquid phase into water; indeed the thermodynamics of protein folding closely follow the behavior of simple hydrophobic molecules in water. Minimizing the number of hydrophobic side-chains exposed to water is an important driving force behind the folding process. Formation of intramolecular hydrogen bonds provides another important contribution to protein stability. The strength of hydrogen bonds depends on their environment, thus H-bonds enveloped in a hydrophobic core contribute more than H-bonds exposed to the aqueous environment to the stability of the native state.

Important intramolecular bonds can be established in a buffer stabilized system of the present disclosure through the addition of water bonders, such as carbohydrates or sugars. In preferred embodiments, the water bonding sugars of the disclosed methods may include, but are not limited to, trehalose, sucrose, glycerol, mannitol, simple sugars, monosaccharides, disaccharides, oligosaccharides, or sugar alcohols like DMSO, ethylene glycol, propylene glycol, and glycerol, as well as sucrose, lactose, maltose, glucose, and polyethylene glycol, hydroxypropyl-β-cyclodextrin (HPβCD), poly(ethylene glycol) (PEG) of different molecular weights, and polymers like carboxylated poly-L-lysine, polyvinylpyrrolidone (PVP), or low molecular weight polyvinyl alcohol and polyglycerol, called X-1000 and Z-1000. In a particularly preferred embodiment, the sugar is trehalose. The incorporation of sugars into the disclosed methods aids in protection of rPA native conformation, alters tonicity, and alters osmolality.

Sugars may be included in the system in various concentrations that can be determined by one of skill in the art. For instance, in certain embodiments of the disclosed methods, the concentration of a sugar will be about 2.5%, about 5%, about 10%, about 15%, or about 20%. Thus, the concentration of a chosen sugar in the disclosed methods may be about 1, about 1.5, about 2, about 2.5, about 3, about 3.5, about 4, about 4.5, about 5, about 5.5, about 6, about 6.5, about 7, about 7.5, about 8, about 8.5, about 9, about 9.5, about 10, about 10.5, about 11, about 11.5, about 12, about 12.5, about 13, about 13.5, about 14, about 14.5, about 15, about 15.5, about 16, about 16.5, about 17, about 17.5, about 18, about 18.5, about 19, about 19.5, about 20, about 20.5, about 21, about 21.5, about 22, about 22.5, about 23, about 23.5, about 24, about 24.5, about 25, about 25.5, about 26.5, about 27, about 27.5, about 28, about 28.5, about 29, about 29.5, about 30, about 30.5, about 31, about 31.5, about 32, about 32.5, about 33, about 33.5, about 34, about 34.5, about 35, about 35.5, about 36, about 36.5, about 37, about 37.5, about 38, about 38.5, about 39, about 39.5, about 40, about 40.5, about 41, about 41.5, about 42, about 42.5, about 43, about 43.5, about 44, about 44.5, about 45, about 45.5, about 46, about 46.5, about 47, about 47.5, about 48, about 48.5, about 49, about 49.5, about 50%, or any amount in-between these values. Alternatively, the sugar can be present in an amount selected from the group consisting of about 2.5% up to about 40%, or any amount in between, such as about 4%, about 5%, about 6%, about 7%, about 8%, about 9%, about 10%, about 12%, about 13%, about 14%, about 15%, about 16%, about 17%, about 18%, about 19%, about 20%, about 25%, about 30%, about 35%, about 40%, about 45% or about 50%, or any amount in-between these values.

B. Buffers

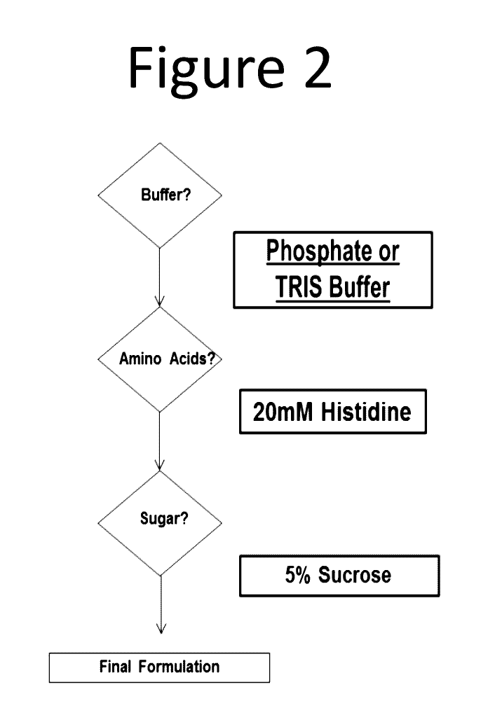

Hydrogen Bonds: Hydrogen bonds are primarily electrostatic in nature and involve an interaction between a hydrogen attached to an electronegative atom and another electronegative acceptor atom (A) that carries a lone pair of electrons. In biological systems the electronegative atoms in both cases are usually nitrogen or oxygen. Many of the hydrogen bonds in proteins occur in networks where each donor participates in multiple interactions with acceptors and each acceptor interacts with multiple donors. This is consistent with the ionic nature of hydrogen bonds in proteins. An example of a proposed stabilization flowchart relating to stabilization of hydrogen bonds is shown in FIG. 2.

Protein stability is the difference in free energy between the unfolded state and the folded state. In the unfolded state the polar components are able to form perfectly satisfactory hydrogen bonds to water that are equivalent to those found in the tertiary structure of the protein. Thus, hydrogen bonding is energetically neutral with respect to protein stability, with the caveat that any absences of hydrogen bonding in a folded protein are thermodynamically highly unfavorable.

Optimal hydrogen bonding and a stabilizing balance of free energy can be established in a buffer stabilized system of the present disclosure through the choice of a buffer. In preferred embodiments, the buffers of the disclosed methods may include, but are not limited to, phosphate buffer saline (PBS) and tris(hydroxymethyl)aminomethane (TRIS). Additional buffers suitable for use in the disclosed stabilizing systems include Bis-TRIS (2-bis[2-hydroxyethyl]amino-2-hydroxymethyl-1,3-propanediol), ADA (N-[2-acetamido]-2-iminodiacetic acid), ACES (2-[2-acetamino]-2-aminoethanesulphonic acid), PIPES (1,4-piperazinediethanesulphonic acid), MOPSO (3[N-morpholino]-2-hydroxypropanesulphonic acid), Bis-TRIS PROPANE (1,3 bis[tris(hydroxymethyl)methylaminopropane]), BES (N,N-bis[2-hydroxyethyl]-2-aminoethanesulphonic acid), MOPS (3[N-morpholino]propanesulphonic acid), TES (2-[2-hydroxy-1,1-bis(hydroxymethyl)ethylamino]ethanesulphonic acid), HEPES (N-[2-hydroxyethyl]piperazine-N′-(2-ethanesulphonic) acid), DIPSO (3-N,N-bis[2-hydroxyethyl]amino-2-hydroxypropanesulphonic) acid), MOBS (4-N-morpholinobutanesulphonic acid), TAPSO (3[N-tris-hydroxymethyl-methylamino]-2-hydroxypropanesulphonic acid), TRIS (2-amino-2-[hydroxymethyl]-1,3-propanediol), HEPPSO (N-[2-hydroxyethyl]piperazine-N′-[2-hydroxypropanesulphonic] acid), POPSO (piperazine-N,N′-bis[2-hydroxypropanesulphonic] acid), TEA (triethanolamine), EPPS (N-[2-hydroxyethyl]-piperazine-N′-[3-propanesulphonic] acid), TRICINE (N-tris[hydroxymethyl]methylglycine), GLY-GLY (diglycine), BICINE (N,N-bis[2-hydroxyethyl]-glycine), HEPBS (N-[2-hydroxyethyl]piperazine-N′-[4-butanesulphonic] acid), TAPS (N-tris[hydroxymethyl]methyl-3-aminopropanesulphonic] acid), AMPD (2-amino-2-methyl-1,3-propanediol), TABS (N-tris[hydroxymethyl]methyl-4-aminobutanesulphonic acid), AMPSO (3-[(1,1-dimethyl-2-hydroxyethyl)amino]-2-hydroxypropanesulphonic acid), CHES (2-(N-cyclohexylamino)ethanesulphonic acid), CAPSO (3-[cyclohexylamino]-2-hydroxy-1-propanesulphonic acid), AMP (2-amino-2-methyl-1-propanol), CAPS (3-cyclohexylamino-1-propanesulphonic acid) or CABS (4-[cyclohexylamino]-1-butanesulphonic acid), preferably AMPD, TABS, AMPSO, CHES, CAPSO, AMP, CAPS or CABS. The choice of the at least one utilized buffer in the disclosed methods and compositions aids in controlling the pH of the system, optimizing solubility based on the Isoelectric Point (pI) of the protein or peptide of interest, and buffering components to control pH (effects the pI). In particularly preferred embodiments, the buffer is a TRIS buffer. The choice of the utilized buffer in the disclosed methods aids in controlling the pH of the system, optimizing solubility based on the Isoelectric Point (pI) of the protein or peptide of interest, and buffering components to control pH (effects the pI).

Buffers included in the disclosed systems may be in various concentrations that can be determined by one of skill in the art. For instance, in certain embodiments of the disclosed methods, the concentration of a buffer will be about 5 mM, about 10 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, about 100 mM, about 105 mM, about 110 mM, about 115 mM, about 120 mM, about 125 mM, about 130 mM, about 135 mM, about 140 mM, about 145 mM, or about 150 mM, or any amount in-between these values. For instance, in exemplary embodiments utilizing a PBS buffer system, the concentration may be about 10 mM PBS. Alternatively, in exemplary embodiments utilizing a TRIS buffer system, the concentration may be about 10 mM TRIS or about 80 mM TRIS.

Additionally, pH of the buffer system is important to achieving and maintaining ideal rPA protein stabilization. Buffers included in the disclosed systems may be set at various pH levels that can be determined by one of skill in the art. For instance, in certain embodiments of the disclosed methods, the pH of a buffer will be about 5, about 5.5, about 6, about 6.5, about 7, about 7.5, about 8, or about 8.5, about 9, about 9.5, or about 10. Thus, the pH of a chosen buffer in the disclosed methods may be 5 about 5.0, about 5.1, about 5.2, about 5.3, about 5.4, about 5.5, about 5.6, about 5.7, about 5.8, about 5.9, about 6.0, 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, about 8.0, about 8.1, about 8.2, about 8.3, about 8.4, about 8.5, about 8.6, about 8.7, about 8.8, about 8.9, about 9.0, about 9.1, about 9.2, about 9.3, about 9.4, about 9.5, about 9.6, about 9.7, about 9.8, about 9.9, or about 10. For instance, in exemplary embodiments utilizing a PBS buffer system, the pH may be about 7.4. Alternatively, in exemplary embodiments utilizing a TRIS buffer system, the pH may be about 8.0.

The disclosed methods and composition can comprise additional buffering agents, such as a pharmaceutically acceptable buffering agent. Examples of buffering agents include, but are not limited to, 2-Amino-2-methyl-1,3-propanediol, ≧99.5% (NT), 2-Amino-2-methyl-1-propanol, ≧99.0% (GC), L-(+)-Tartaric acid, ≧99.5% (T), ACES, ≧99.5% (T), ADA, ≧99.0% (T), Acetic acid, ≧99.5% (GC/T), Acetic acid, for luminescence, ≧99.5% (GC/T), Ammonium acetate solution, for molecular biology, ˜5 M in H₂O, Ammonium acetate, for luminescence, ≧99.0% (calc. on dry substance, T), Ammonium bicarbonate, ≧99.5% (T), Ammonium citrate dibasic, ≧99.0% (T), Ammonium formate solution, 10 M in H₂O, Ammonium formate, ≧99.0% (calc. based on dry substance, NT), Ammonium oxalate monohydrate, ≧99.5% (RT), Ammonium phosphate dibasic solution, 2.5 M in H₂O, Ammonium phosphate dibasic, ≧99.0% (T), Ammonium phosphate monobasic solution, 2.5 M in H₂O, Ammonium phosphate monobasic, ≧99.5% (T), Ammonium sodium phosphate dibasic tetrahydrate, ≧99.5% (NT), Ammonium sulfate solution, for molecular biology, 3.2 M in H₂O, Ammonium tartrate dibasic solution, 2 M in H₂O (colorless solution at 20° C.), Ammonium tartrate dibasic, ≧99.5% (T), BES buffered saline, for molecular biology, 2× concentrate, BES, ≧99.5% (T), BES, for molecular biology, ≧99.5% (T), BICINE buffer Solution, for molecular biology, 1 M in H₂O, BICINE, ≧99.5% (T), BIS-TRIS, ≧99.0% (NT), Bicarbonate buffer solution, ≧0.1 M Na₂CO₃, ≧0.2 M NaHCO₃, Boric acid, ≧99.5% (T), Boric acid, for molecular biology, ≧99.5% (T), CAPS, ≧99.0% (TLC), CHES, ≧99.5% (T), Calcium acetate hydrate, ≧99.0% (calc. on dried material, KT), Calcium carbonate, precipitated, ≧99.0% (KT), Calcium citrate tribasic tetrahydrate, ≧98.0% (calc. on dry substance, KT), Citrate Concentrated Solution, for molecular biology, 1 M in H₂O, Citric acid, anhydrous, ≧99.5% (T), Citric acid, for luminescence, anhydrous, ≧99.5% (T), Diethanolamine, ≧99.5% (GC), EPPS, ≧99.0% (T), Ethylenediaminetetraacetic acid disodium salt dihydrate, for molecular biology, ≧99.0% (T), Formic acid solution, 1.0 M in H₂O, Gly-Gly-Gly, ≧99.0% (NT), Gly-Gly, ≧99.5% (NT), Glycine, ≧99.0% (NT), Glycine, for luminescence, ≧99.0% (NT), Glycine, for molecular biology, ≧99.0% (NT), HEPES buffered saline, for molecular biology, 2× concentrate, HEPES, ≧99.5% (T), HEPES, for molecular biology, ≧99.5% (T), Imidazole buffer Solution, 1 M in H₂O, Imidazole, ≧99.5% (GC), Imidazole, for luminescence, ≧99.5% (GC), Imidazole, for molecular biology, ≧99.5% (GC), Lipoprotein Refolding Buffer, Lithium acetate dihydrate, ≧99.0% (NT), Lithium citrate tribasic tetrahydrate, ≧99.5% (NT), MES hydrate, ≧99.5% (T), MES monohydrate, for luminescence, ≧99.5% (T), MES solution, for molecular biology, 0.5 M in H₂O, MOPS, ≧99.5% (T), MOPS, for luminescence, ≧99.5% (T), MOPS, for molecular biology, ≧99.5% (T), Magnesium acetate solution, for molecular biology, ˜1 M in H₂O, Magnesium acetate tetrahydrate, ≧99.0% (KT), Magnesium citrate tribasic nonahydrate, ≧98.0% (calc. based on dry substance, KT), Magnesium formate solution, 0.5 M in H₂O, Magnesium phosphate dibasic trihydrate, ≧98.0% (KT), Neutralization solution for the in-situ hybridization for in-situ hybridization, for molecular biology, Oxalic acid dihydrate, ≧99.5% (RT), PIPES, ≧99.5% (T), PIPES, for molecular biology, ≧99.5% (T), Phosphate buffered saline, solution (autoclaved), Phosphate buffered saline, washing buffer for peroxidase conjugates in Western Blotting, 10× concentrate, Piperazine, anhydrous, ≧99.0% (T), Potassium D-tartrate monobasic, ≧99.0% (T), Potassium acetate solution, for molecular biology, Potassium acetate solution, for molecular biology, 5 M in H₂O, Potassium acetate solution, for molecular biology, ˜1 M in H₂O, Potassium acetate, ≧99.0% (NT), Potassium acetate, for luminescence, ≧99.0% (NT), Potassium acetate, for molecular biology, ≧99.0% (NT), Potassium bicarbonate, ≧99.5% (T), Potassium carbonate, anhydrous, ≧99.0% (T), Potassium chloride, ≧99.5% (AT), Potassium citrate monobasic, ≧99.0% (dried material, NT), Potassium citrate tribasic solution, 1 M in H₂O, Potassium formate solution, 14 M in H₂O, Potassium formate, ≧99.5% (NT), Potassium oxalate monohydrate, ≧99.0% (RT), Potassium phosphate dibasic, anhydrous, ≧99.0% (T), Potassium phosphate dibasic, for luminescence, anhydrous, ≧99.0% (T), Potassium phosphate dibasic, for molecular biology, anhydrous, ≧99.0% (T), Potassium phosphate monobasic, anhydrous, ≧99.5% (T), Potassium phosphate monobasic, for molecular biology, anhydrous, ≧99.5% (T), Potassium phosphate tribasic monohydrate, ≧95% (T), Potassium phthalate monobasic, ≧99.5% (T), Potassium sodium tartrate solution, 1.5 M in H₂O, Potassium sodium tartrate tetrahydrate, ≧99.5% (NT), Potassium tetraborate tetrahydrate, ≧99.0% (T), Potassium tetraoxalate dihydrate, ≧99.5% (RT), Propionic acid solution, 1.0 M in H₂O, STE buffer solution, for molecular biology, pH 7.8, STET buffer solution, for molecular biology, pH 8.0, Sodium 5,5-diethylbarbiturate, ≧99.5% (NT), Sodium acetate solution, for molecular biology, ˜3 M in H₂O, Sodium acetate trihydrate, ≧99.5% (NT), Sodium acetate, anhydrous, ≧99.0% (NT), Sodium acetate, for luminescence, anhydrous, ≧99.0% (NT), Sodium acetate, for molecular biology, anhydrous, ≧99.0% (NT), Sodium bicarbonate, ≧99.5% (T), Sodium bitartrate monohydrate, ≧99.0% (T), Sodium carbonate decahydrate, ≧99.5% (T), Sodium carbonate, anhydrous, ≧99.5% (calc. on dry substance, T), Sodium citrate monobasic, anhydrous, ≧99.5% (T), Sodium citrate tribasic dihydrate, ≧99.0% (NT), Sodium citrate tribasic dihydrate, for luminescence, ≧99.0% (NT), Sodium citrate tribasic dihydrate, for molecular biology, ≧99.5% (NT), Sodium formate solution, 8 M in H₂O, Sodium oxalate, ≧99.5% (RT), Sodium phosphate dibasic dihydrate, ≧99.0% (T), Sodium phosphate dibasic dihydrate, for luminescence, ≧99.0% (T), Sodium phosphate dibasic dihydrate, for molecular biology, ≧99.0% (T), Sodium phosphate dibasic dodecahydrate, ≧99.0% (T), Sodium phosphate dibasic solution, 0.5 M in H₂O, Sodium phosphate dibasic, anhydrous, ≧99.5% (T), Sodium phosphate dibasic, for molecular biology, ≧99.5% (T), Sodium phosphate monobasic dihydrate, ≧99.0% (T), Sodium phosphate monobasic dihydrate, for molecular biology, ≧99.0% (T), Sodium phosphate monobasic monohydrate, for molecular biology, ≧99.5% (T), Sodium phosphate monobasic solution, 5 M in H₂O, Sodium pyrophosphate dibasic, ≧99.0% (T), Sodium pyrophosphate tetrabasic decahydrate, ≧99.5% (T), Sodium tartrate dibasic dihydrate, ≧99.0% (NT), Sodium tartrate dibasic solution, 1.5 M in H₂O (colorless solution at 20° C.), Sodium tetraborate decahydrate, ≧99.5% (T), TAPS, ≧99.5% (T), TES, ≧99.5% (calc. based on dry substance, T), TM buffer solution, for molecular biology, pH 7.4, TNT buffer solution, for molecular biology, pH 8.0, TRIS Glycine buffer solution, 10× concentrate, TRIS acetate-EDTA buffer solution, for molecular biology, TRIS buffered saline, 10× concentrate, TRIS glycine SDS buffer solution, for electrophoresis, 10× concentrate, TRIS phosphate-EDTA buffer solution, for molecular biology, concentrate, 10× concentrate, Tricine, ≧99.5% (NT), Triethanolamine, ≧99.5% (GC), Triethylamine, ≧99.5% (GC), Triethylammonium acetate buffer, volatile buffer, ˜1.0 M in H₂O, Triethylammonium phosphate solution, volatile buffer, ˜1.0 M in H₂O, Trimethylammonium acetate solution, volatile buffer, ˜1.0 M in H₂O, Trimethylammonium phosphate solution, volatile buffer, ˜1 M in H₂O, Tris-EDTA buffer solution, for molecular biology, concentrate, 100× concentrate, Tris-EDTA buffer solution, for molecular biology, pH 7.4, Tris-EDTA buffer solution, for molecular biology, pH 8.0, Trizma® acetate, ≧99.0% (NT), Trizma® base, ≧99.8% (T), Trizma® base, ≧99.8% (T), Trizma® base, for luminescence, ≧99.8% (T), Trizma® base, for molecular biology, ≧99.8% (T), Trizma® carbonate, ≧98.5% (T), Trizma® hydrochloride buffer solution, for molecular biology, pH 7.2, Trizma® hydrochloride buffer solution, for molecular biology, pH 7.4, Trizma® hydrochloride buffer solution, for molecular biology, pH 7.6, Trizma® hydrochloride buffer solution, for molecular biology, pH 8.0, Trizma® hydrochloride, ≧99.0% (AT), Trizma® hydrochloride, for luminescence, ≧99.0% (AT), Trizma® hydrochloride, for molecular biology, ≧99.0% (AT), and Trizma® maleate, ≧99.5% (NT).

C. Reducing Agents

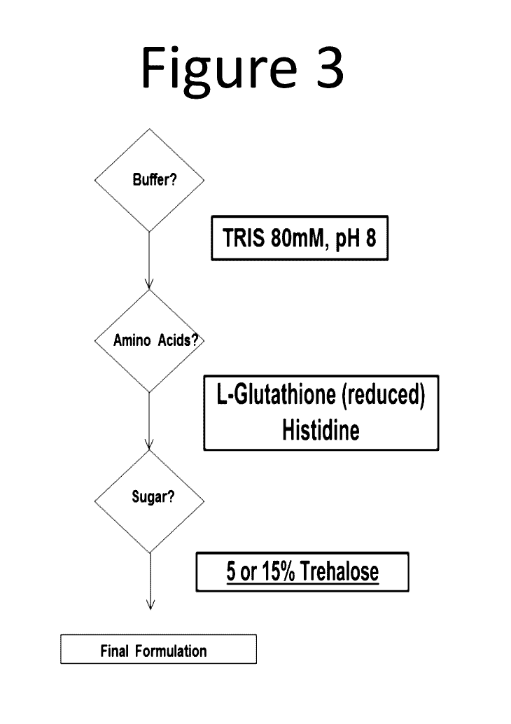

Disulfide Bonds: Many extracellular proteins contain disulfide bonds. In these proteins the presence of disulfide bonds adds considerable stability to the folded state where in many cases reduction of the cystine linkages is sufficient to induce unfolding. The source of the stability appears to be entropic rather than enthalpic. The introduction of a disulfide bond reduces the entropy of the unfolded state by reducing the degrees of freedom available to the disordered polypeptide chain. This stabilizes the folded state by decreasing the entropy difference between the folded and unfolded state. An example of a proposed stabilization flowchart relating to stabilization of disulfide bonds is shown in FIG. 3.

Important disulfide bonds can be strengthened or established in a buffer stabilized system of the present disclosure through the addition of reducing. Reducing agents suitable for use in the disclosed stabilizing systems include, but are not limited to, pharmaceutically acceptable reducing agent like cysteine, glutathione, a combination of glutathione and glutathione S-transferase, Dithiothreitol (DTT), cysteamine, thioredoxin, N-acetyl-L-cysteine (NAC), alpha-lipoic acid, 2-mercaptoethanol, 2-mercaptoethanesulfonic acid, mercapto-propionyglycine, tris(2-carboxyethyl)phophine (TCEP) and combinations thereof. EDTA, as a chelating agent, may inhibit the metal-catalyzed oxidation of the sulfhydryl groups, thus reducing the formation of disulfide-linked aggregates. A preferred concentration of EDTA is 0.001-0.5%, more preferably 0.005-0.4%, more preferably 0.0075-0.3%, or even more preferably 0.01-0.2%.

D. Salts

Ionic Interactions: The association of two oppositely charged ionic groups in a protein is known as a salt bridge or ion pair and is a common feature of most proteins. Typically these interactions contribute very little to rPA protein stability since the isolated ionic groups are so effectively solvated by water. As a consequence very few un-solvated salt bridges are found in the interior of proteins.

Important ionic interactions can be strengthened or established in a buffer stabilized system of the present disclosure through the addition of salts. In preferred embodiments, the salts utilized in the disclosed methods may include, but are not limited to, sodium chloride, sodium succinate, sodium sulfate, potassium chloride, magnesium chloride, magnesium sulfate, and calcium chloride. The incorporation of salts into the disclosed methods aids in increasing the surface tension of water ionic strength and optimizing ionic strength, particularly in instances when stabilizing an ion-dependent folding of the protein domain (e.g. rPA has calcium-dependent binding domains).

Salts may function as tonicity modifiers, which contributes to the isotonicity of the formulations, and may be added to the disclosed compositions. The tonicity modifier useful for the present invention include the salts listed above.

One or more salts may be included in the disclosed systems in various concentrations that can be determined by one of skill in the art. For instance, in certain embodiments of the disclosed methods, the concentration of calcium chloride will be about 10 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, about 100 mM, about 105 mM, about 110 mM, about 115 mM, about 120 mM, about 125 mM, about 130 mM, about 135 mM, about 140 mM, about 145 mM, about 150 mM, about 155 mM, about 160 mM, about 165 mM, about 170 mM, about 175 mM, about 180 mM, about 185 mM, about 190 mM, about 195 mM, about 200 mM, or any amount in-between these values. For instance, in exemplary embodiments utilizing a sodium chloride, the concentration may be about 100-about 150 mM. In exemplary embodiments utilizing calcium chloride, the concentration may be about 100-about 150 mM. Thus, for example, the concentration of a chosen salt in the disclosed methods may be about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, about 101, about 102, about 103, about 104, about 105, about 106, about 107, about 108, about 109, about 110, about 111, about 112, about 113, about 114, about 115, about 116, about 117, about 118, about 119, about 120, about 121, about 122, about 123, about 124, about 125, about 126, about 127, about 128, about 129, about 130, about 131, about 132, about 133, about 134, about 135, about 136, about 137, about 138, about 139, about 140, about 141, about 142, about 143, about 144, about 145, about 146, about 147, about 148, about 149, about 150, about 151, about 152, about 153, about 154, about 155, about 156, about 157, about 158, about 159, about 160, about 161, about 162, about 163, about 164, about 165, about 166, about 167, about 168, about 169, about 170, about 171, about 172, about 173, about 174, about 175, about 176, about 177, about 178, about 179, about 180, about 181, about 182, about 183, about 184, about 185, about 186, about 187, about 188, about 189, about 190, about 191, about 192, about 193, about 194, about 195, about 196, about 197, about 198, about 199, about 200 mM, or any amount in-between these values. In exemplary embodiments utilizing magnesium chloride, the concentration may be about 1 about 150 mM. Thus, for example, the concentration of a chosen salt in the disclosed methods may be about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100 mM, about 101, about 102, about 103, about 104, about 105, about 106, about 107, about 108, about 109, about 110, about 111, about 112, about 113, about 114, about 115, about 116, about 117, about 118, about 119, about 120, about 121, about 122, about 123, about 124, about 125, about 126, about 127, about 128, about 129, about 130, about 131, about 132, about 133, about 134, about 135, about 136, about 137, about 138, about 139, about 140, about 141, about 142, about 143, about 144, about 145, about 146, about 147, about 148, about 149, about 150, or any amount in-between these values.

Preferred salts for this invention include NaCl and MgCl₂. A preferred concentration of NaCl is about 75-150 mM. A preferred concentration of MgCl₂ is about 1-150 mM.

E. Amino Acids

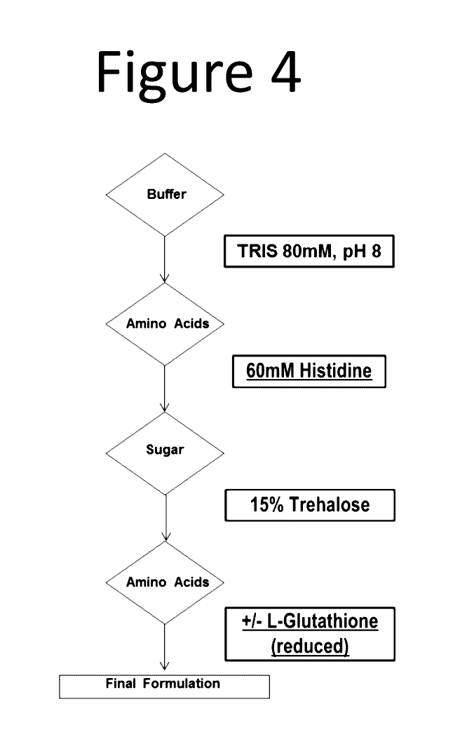

Dipole-Dipole Interactions: Dipole-dipole interactions are weak interactions that arise from the close association of permanent or induced dipoles. Collectively these forces are known as Van der Waals interactions. Proteins contain a large number of these interactions, which vary considerably in strength. The strongest interactions are observed between permanent dipoles and are an important feature of the peptide bond. London or dispersion forces are the weakest of all of the dipole-dipole. As a group, the Van der Waals forces are important for stabilizing interactions between proteins and their complementary ligands whether the ligands are proteins or small molecules. An example of a proposed stabilization flowchart relating to stabilization of dipole-dipole interactions is shown in FIG. 4.

Important dipole-dipole interactions can be strengthened or established in a buffer stabilized system of the present disclosure through the addition of amino acids. In preferred embodiments, the amino acids utilized in the disclosed methods may include, but are not limited to, alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine. Modified and/or synthetic forms of amino acids can also be utilized in the methods and compositions of the invention. Modified and/or synthetic forms of amino acids can also be utilized in the methods and compositions of the disclosure, for example, non-naturally encoded amino acids include, but are not limited to, an unnatural analogue of a tyrosine amino acid; an unnatural analogue of a glutamine amino acid; an unnatural analogue of a phenylalanine amino acid; an unnatural analogue of a serine amino acid; an unnatural analogue of a threonine amino acid; an alkyl, aryl, acyl, azido, cyano, halo, hydrazine, hydrazide, hydroxyl, alkenyl, alkynl, ether, thiol, sulfonyl, seleno, ester, thioacid, borate, boronate, phospho, phosphono, phosphine, heterocyclic, enone, imine, aldehyde, hydroxylamine, keto, or amino substituted amino acid, or any combination thereof; an amino acid with a photoactivatable cross-linker; a spin-labeled amino acid; a fluorescent amino acid; an amino acid with a novel functional group; an amino acid that covalently or noncovalently interacts with another molecule; a metal binding amino acid; a metal-containing amino acid; a radioactive amino acid; a photocaged and/or photoisomerizable amino acid; a biotin or biotin-analogue containing amino acid; a glycosylated or carbohydrate modified amino acid; a keto containing amino acid; amino acids comprising polyethylene glycol or polyether; a heavy atom substituted amino acid; a chemically cleavable or photocleavable amino acid; an amino acid with an elongated side chain; an amino acid containing a toxic group; a sugar substituted amino acid, e.g., a sugar substituted serine or the like; a carbon-linked sugar-containing amino acid; a redox-active amino acid; an α-hydroxy containing acid; an amino thio acid containing amino acid; an α,α di-substituted amino acid; a β-amino acid; and a cyclic amino acid other than proline. In particularly preferred embodiments, the amino acid may be histidine, glutathione, or alanine. In an even more preferred embodiment, the amino acid is histidine. The incorporation of amino acids into the disclosed methods aids in directing protein binding, buffering capacity, and antioxidant properties, as well as suppressing the aggregation of folding intermediates, radical attacks by reactive oxygen and nitrogen species, and preventing denaturation.

Like the salts discussed above, amino acids can also be considered tonicity modifiers. Amino acids that are pharmaceutically acceptable and suitable for this purpose include proline, alanine, L-arginine, asparagine, L-aspartic acid, glycine, serine, lysine, and histidine. A preferred amino acid for this invention is histidine. A preferred concentration of histidine is roughly 5-80 mM.

Amino acids may be included in the disclosed systems in various concentrations that can be determined by one of skill in the art. For instance, in certain embodiments of the disclosed methods, the concentration of an amino acid will be about 5 mM, about 10 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, about 100 mM, or any amount in-between these values. For instance, in exemplary embodiments utilizing a glutathione, the concentration may be about 16 mM glutathione. In exemplary embodiments utilizing histidine, the concentration may be about 20 mM or about 60 mM histidine. In exemplary embodiments utilizing a alanine, the concentration may be about 10 mM alanine. Thus, the concentration of a chosen amino acid in the disclosed methods may be about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100 mM, or any amount in-between these values.

F. Additional Ingredients

Additional compounds suitable for use in the disclosed methods or compositions include, but are not limited to, one or more solvents, such as an organic phosphate-based solvent, bulking agents, coloring agents, pharmaceutically acceptable excipients, a preservative, pH adjuster, buffer, chelating agent, etc. The additional compounds can be admixed into a previously formulated composition, or the additional compounds can be added to the original mixture to be further formulated. In certain of these embodiments, one or more additional compounds are admixed into an existing disclosed composition immediately prior to its use.

Suitable preservatives in the disclosed composition include, but are not limited to, cetylpyridinium chloride, benzalkonium chloride, benzyl alcohol, chlorhexidine, imidazolidinyl urea, phenol, potassium sorbate, benzoic acid, bronopol, chlorocresol, paraben esters, phenoxyethanol, sorbic acid, alpha-tocophernol, ascorbic acid, ascorbyl palmitate, butylated hydroxyanisole, butylated hydroxytoluene, sodium ascorbate, sodium metabisulphite, citric acid, edetic acid, semi-synthetic derivatives thereof, and combinations thereof. Other suitable preservatives include, but are not limited to, benzyl alcohol, chlorhexidine (bis (p-chlorophenyldiguanido) hexane), chlorphenesin (3-(-4-chloropheoxy)-propane-1,2-diol), Kathon CG (methyl and methylchloroisothiazolinone), parabens (methyl, ethyl, propyl, butyl hydrobenzoates), phenoxyethanol (2-phenoxyethanol), sorbic acid (potassium sorbate, sorbic acid), Phenonip (phenoxyethanol, methyl, ethyl, butyl, propyl parabens), Phenoroc (phenoxyethanol 0.73%, methyl paraben 0.2%, propyl paraben 0.07%), Liquipar Oil (isopropyl, isobutyl, butylparabens), Liquipar PE (70% phenoxyethanol, 30% liquipar oil), Nipaguard MPA (benzyl alcohol (70%), methyl & propyl parabens), Nipaguard MPS (propylene glycol, methyl & propyl parabens), Nipasept (methyl, ethyl and propyl parabens), Nipastat (methyl, butyl, ethyl and propyel parabens), Elestab 388 (phenoxyethanol in propylene glycol plus chlorphenesin and methylparaben), and Killitol (7.5% chlorphenesin and 7.5% methyl parabens).

The disclosed composition may further comprise at least one pH adjuster. Suitable pH adjusters in the disclosed composition include, but are not limited to, diethyanolamine, lactic acid, monoethanolamine, triethylanolamine, sodium hydroxide, sodium phosphate, semi-synthetic derivatives thereof, and combinations thereof.

In addition, the disclosed composition can comprise a chelating agent. In one embodiment of the disclosed, the chelating agent is present in an amount of about 0.0005% to about 1%. Examples of chelating agents include, but are not limited to, ethylenediamine, ethylenediaminetetraacetic acid (EDTA), phytic acid, polyphosphoric acid, citric acid, gluconic acid, acetic acid, lactic acid, and dimercaprol, and a preferred chelating agent is ethylenediaminetetraacetic acid.

The disclosed methods and compositions can comprise one or more emulsifying agents to aid in the formation of emulsions. Emulsifying agents include compounds that aggregate at the oil/water interface to form a kind of continuous membrane that prevents direct contact between two adjacent droplets. Certain embodiments of the present disclosure feature nanoemulsion compositions that may readily be diluted with water or another aqueous phase to a desired concentration without impairing their desired properties.

### III. Buffer-Stabilized rPA Protein Compositions

The compositions of the invention comprise rPA protein combined with a protein-stabilizing buffer system. The present disclosure is directed, in part, to novel, optimized rPA compositions to stabilize the secondary and tertiary structures of rPA by proactively screening and addressing all of the destabilizing or un-stabilizing factors that would affect the rPA protein structure and lead to aggregation and/or degradation of the rPA protein.

The disclosed buffer stabilized rPA protein compositions comprise rPA protein, a buffer, a salt, a sugar, an antioxidant, an amino acid, or a combination thereof. Exemplary components (i.e. buffers, salts, sugars, antioxidants, and amino acids) are disclosed throughout the specification and the examples. The disclosed compositions have been demonstrated to unexpected stabilize rPA protein in solution over extended periods of time, even when introduced to stressor that can potentially cause denaturation or aggregation, such as heat.

In one embodiment of the disclosed composition, the stabilizing buffer system comprises: (1) a TRIS (tris(hydroxymethyl)aminomethane) buffer; (2) at least one salt, such as sodium chloride or calcium chloride; (3) at least one sugar, such as trehalose and sucrose; (4) at least one amino acid, such as histidine, alanine, or glutathione; or (5) any combination thereof.

In some embodiments, the pH of composition is between about 5 to about 10, between about 6 to about 9, or between about 7 to about 8. For instance, the pH of a disclosed buffer stabilized composition may be about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, about 8.0, about 8.1, about 8.2, about 8.3, about 8.4, about 8.5, about 8.6, about 8.7, about 8.8, about 8.9, about 9.0, about 9.1, about 9.2, about 9.3, about 9.4, about 9.5, about 9.6, about 9.7, about 9.8, about 9.9, about 10, or any amount in-between these values.

In another embodiment, the disclosed rPA compositions comprise at least one sugar. Preferred sugars include, but are not limited to, trehalose and sucrose. In preferred embodiments, the sugar can be trehalose. The sugar can be present in an amount selected from the group consisting of about 2.5% up to about 40%, or any amount in between, such as about 4%, about 5%, about 6%, about 7%, about 8%, about 9%, about 10%, about 12%, about 13%, about 14%, about 15%, about 16%, about 17%, about 18%, about 19%, about 20%, about 25%, about 30%, about 35%, or about 45%. In other embodiments of the disclosed compositions, the concentration of a sugar will be about 2.5%, about 5%, about 10%, about 15%, or about 20%. Thus, the concentration of a chosen sugar in the disclosed methods may be about 2.5%, about 5%, about 10%, about 15%, or about 20%. Thus, the concentration of a chosen sugar in the disclosed methods may be about 1, about 1.5, about 2, about 2.5, about 3, about 3.5, about 4, about 4.5, about 5, about 5.5, about 6, about 6.5, about 7, about 7.5, about 8, about 8.5, about 9, about 9.5, about 10, about 10.5, about 11, about 11.5, about 12, about 12.5, about 13, about 13.5, about 14, about 14.5, about 15, about 15.5, about 16, about 16.5, about 17, about 17.5, about 18, about 18.5, about 19, about 19.5, about 20, about 20.5, about 21, about 21.5, about 22, about 22.5, about 23, about 23.5, about 24, about 24.5, about 25%, or any amount in-between these values.

One or more salts may be included in the disclosed systems (e.g., methods and compositions) in various concentrations that can be determined by one of skill in the art. For instance, in certain embodiments of the disclosed compositions, the concentration of an amino acid will be about 50 mM, about 55 mM, about 60 mM, about 65 mM, about 70 mM, about 75 mM, about 80 mM, about 85 mM, about 90 mM, about 95 mM, about 100 mM, about 105 mM, about 110 mM, about 115 mM, about 120 mM, about 125 mM, about 130 mM, about 135 mM, about 140 mM, about 145 mM, about 150 mM, about 155 mM, about 160 mM, about 165 mM, about 170 mM, about 175 mM, about 180 mM, about 185 mM, about 190 mM, about 195 mM, or about 200 mM. For instance, in exemplary embodiments utilizing a sodium chloride, the concentration may be about 100-about 150 mM. In exemplary embodiments utilizing calcium chloride, the concentration may be about 100-about 150 mM. Thus, the concentration of a chosen salt in the disclosed compositions may be about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, about 101, about 102, about 103, about 104, about 105, about 106, about 107, about 108, about 109, about 110, about 111, about 112, about 113, about 114, about 115, about 116, about 117, about 118, about 119, about 120, about 121, about 122, about 123, about 124, about 125, about 126, about 127, about 128, about 129, about 130, about 131, about 132, about 133, about 134, about 135, about 136, about 137, about 138, about 139, about 140, about 141, about 142, about 143, about 144, about 145, about 146, about 147, about 148, about 149, about 150, about 151, about 152, about 153, about 154, about 155, about 156, about 157, about 158, about 159, about 160, about 161, about 162, about 163, about 164, about 165, about 166, about 167, about 168, about 169, about 170, about 171, about 172, about 173, about 174, about 175, about 176, about 177, about 178, about 179, about 180, about 181, about 182, about 183, about 184, about 185, about 186, about 187, about 188, about 189, about 190, about 191, about 192, about 193, about 194, about 195, about 196, about 197, about 198, about 199, about 200 mM, or any amount in-between these values.

Important dipole-dipole interactions can be strengthened or established in a buffer stabilized system of the present disclosure through the addition of amino acids. In preferred embodiments, the amino acids utilized in the disclosed methods may include, but are not limited to, alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine. Modified, synthetic, or semi-synthetic forms of amino acids can also be used in the methods or compositions of the invention. Modified and/or synthetic forms of amino acids can also be utilized in the methods and compositions of the disclosure, for example, non-naturally encoded amino acids include, but are not limited to, an unnatural analogue of a tyrosine amino acid; an unnatural analogue of a glutamine amino acid; an unnatural analogue of a phenylalanine amino acid; an unnatural analogue of a serine amino acid; an unnatural analogue of a threonine amino acid; an alkyl, aryl, acyl, azido, cyano, halo, hydrazine, hydrazide, hydroxyl, alkenyl, alkynl, ether, thiol, sulfonyl, seleno, ester, thioacid, borate, boronate, phospho, phosphono, phosphine, heterocyclic, enone, imine, aldehyde, hydroxylamine, keto, or amino substituted amino acid, or any combination thereof; an amino acid with a photoactivatable cross-linker; a spin-labeled amino acid; a fluorescent amino acid; an amino acid with a novel functional group; an amino acid that covalently or noncovalently interacts with another molecule; a metal binding amino acid; a metal-containing amino acid; a radioactive amino acid; a photocaged and/or photoisomerizable amino acid; a biotin or biotin-analogue containing amino acid; a glycosylated or carbohydrate modified amino acid; a keto containing amino acid; amino acids comprising polyethylene glycol or polyether; a heavy atom substituted amino acid; a chemically cleavable or photocleavable amino acid; an amino acid with an elongated side chain; an amino acid containing a toxic group; a sugar substituted amino acid, e.g., a sugar substituted serine or the like; a carbon-linked sugar-containing amino acid; a redox-active amino acid; an α-hydroxy containing acid; an amino thio acid containing amino acid; an α,α di-substituted amino acid; a β-amino acid; and a cyclic amino acid other than proline. In particularly preferred embodiments, the amino acid may be histidine, glutathione, or alanine. In even more preferred embodiments, the amino acid can be histidine. The incorporation of amino acids into the disclosed compositions aids in directing rPA protein binding, buffering capacity, and antioxidant properties, as well as suppressing the aggregation of folding intermediates, radical attacks by reactive oxygen and nitrogen species, and preventing denaturation.

Amino acids may be included in the disclosed systems in various concentrations that can be determined by one of skill in the art. For instance, in certain embodiments of the disclosed methods, the concentration of an amino acid will be about 5 mM, about 10 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, or about 100 mM. For instance, in exemplary embodiments utilizing a glutathione, the concentration may be about 16 mM glutathione. In exemplary embodiments utilizing histidine, the concentration may be about 20 mM or about 60 mM histidine. In exemplary embodiments utilizing a alanine, the concentration may be about 10 mM alanine. Thus, the concentration of a chosen amino acid in the disclosed compositions may be about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100 mM, or any amount in-between these values.

Additional compounds suitable for use in the disclosed compositions include, but are not limited to, one or more solvents, such as an organic phosphate-based solvent, bulking agents, coloring agents, pharmaceutically acceptable excipients, a preservative, pH adjuster, buffer, chelating agent, etc. The additional compounds can be admixed into a previously formulated composition, or the additional compounds can be added to the original mixture to be further formulated. In certain of these embodiments, one or more additional compounds are admixed into an existing disclosed composition immediately prior to its use. Such additional ingredients include, but are not limited to, those listed above in Section III—Novel Methods to Stabilize rPA Protein.

In some embodiments, the disclosed buffer stabilized compositions will further comprise at least one reducing agent. Reducing agents suitable for use in the disclosed composition are known in the art, and can be important for strengthening or establishing disulfide bonds in a buffer stabilized system. Reducing agents suitable for use in the disclosed stabilizing systems include, but are not limited to, pharmaceutically acceptable reducing agent like cysteine, glutathione, a combination of glutathione and glutathione S-transferase, Dithiothreitol (DTT), cysteamine, thioredoxin, N-acetyl-L-cysteine (NAC), alpha-lipoic acid, 2-mercaptoethanol, 2-mercaptoethanesulfonic acid, mercapto-propionyglycine, tris(2-carboxyethyl)phophine (TCEP) and combinations thereof. EDTA, as a chelating agent, may inhibit the metal-catalyzed oxidation of the sulfhydryl groups, thus reducing the formation of disulfide-linked aggregates. A preferred concentration of EDTA is 0.001-0.5%, more preferably 0.005-0.4%, more preferably 0.0075-0.3%, or even more preferably 0.01-0.2%.

Stability of the rPA protein can be evaluated by one or more of the following factors: (1) evaluating the physical, chemical, and/or biological stability of the rPA protein; (2) determining whether rPA protein aggregates or particulates are present; (3) determining whether the rPA protein is susceptible to or undergoing denaturation; (4) evaluating the thermostability of the rPA protein by exposing the proteins to an elevated temperature and determining whether the rPA protein denatures or changes in concentration by more than about 10%, about 15%, about 20%, about 25%, about 30%, about 35%, about 40%, about 45%, or about 50%; (5) measuring rPA protein concentration to determine if the concentration changes over time, demonstrating rPA protein instability. For example, if the rPA protein concentration changes by more than 10%, about 15%, about 20%, about 25%, about 30%, about 35%, about 40%, about 45%, or about 50% over time, then this is evidence of rPA protein instability; (6) evaluating the color of a disclosed composition comprising a stabilized rPA protein, where a white to off white color is acceptable and a yellow (light to dark), tan, and shades of brown are not acceptable as the indicate rPA protein instability; and/or (7) evaluating a nanoemulsion composition comprising a stabilized rPA protein to determine if the nanoemulsion particle size changes significantly over time, which is evidence of an unstable composition (e.g., changes by more than about 10%, about 15%, about 20%, about 25%, about 30%, about 35%, about 40%, about 45%, or about 50% time time).

### IV. rPA Pharmaceutical Compositions

The buffer-stabilized rPA protein compositions of the present disclosure may be formulated into rPA pharmaceutical compositions, such as a vaccine, that are administered in a therapeutically effective amount to a subject and may further comprise suitable, pharmaceutically-acceptable excipients, additives, or preservatives. Suitable excipients, additives, and preservatives are well known in the art.

By the phrase “therapeutically effective amount” it is meant any amount of the rPA composition that is effective in preventing, treating, or ameliorating a disease, pathogen, malignancy, or condition associated with the rPA protein or antigen present in the buffer-stabilized composition. By “protective immune response” it is meant that the immune response is associated with prevention, treating, or amelioration of a disease. Complete prevention is not required, though is encompassed by the present disclosure. The immune response can be evaluated using the methods discussed herein or by any method known by a person of skill in the art.

The rPA pharmaceutical compositions may be formulated for immediate release, sustained release, controlled release, delayed release, or any combinations thereof, into the epidermis or dermis.

An agent of the present disclosure can be administered for therapy by any suitable route of administration. It will also be appreciated that the preferred route will vary with the condition and age of the recipient, and the disease being treated.

For instance, the rPA compositions can be administered by oral, parenteral (e.g., intramuscular, intraperitoneal, intravenous, ICV, intracisternal injection or infusion, subcutaneous injection, or implant), by inhalation spray nasal, vaginal, rectal, sublingual, urethral (e.g., urethral suppository) or topical routes of administration (e.g., gel, ointment, cream, aerosol, etc.) and can be formulated, alone or together, in suitable dosage unit formulations containing conventional non-toxic pharmaceutically acceptable carriers, adjuvants, excipients, and vehicles appropriate for each route of administration. Non-limiting examples of carriers include phosphate buffered saline (PBS), saline or a biocompatible matrix material such as a decellularized liver matrix (DCM as disclosed in Wang et al. (2014) J. Biomed. Mater Res. A. 102(4):1017-1025) for topical or local administration. The compositions can optionally contain a protease inhibitor, glycerol and/or dimethyl sulfoxide (DMSO).

The rPA compositions can be conveniently presented in dosage unit form and can be prepared by any of the methods well known in the art of pharmacy. The rPA compositions can be, for example, prepared by uniformly and intimately bringing the active ingredient into association with a liquid carrier, a finely divided solid carrier or both, and then, if necessary, shaping the product into the desired formulation. In the composition the rPA protein or peptide is included in an amount sufficient to produce the desired therapeutic effect. For example, rPA pharmaceutical compositions of the disclosure may take a form suitable for virtually any mode of administration, including, for example, topical, ocular, oral, buccal, systemic, nasal, injection, transdermal, rectal, and vaginal, or a form suitable for administration by inhalation or insufflation.

Intranasal administration is a particularly preferred mode of administration that includes administration via the nose, either with or without concomitant inhalation during administration. Such administration is typically through contact by the pharmaceutical composition comprising the composition with the nasal mucosa, nasal turbinates or sinus cavity. Administration by inhalation comprises intranasal administration, or may include oral inhalation. Such administration may also include contact with the oral mucosa, bronchial mucosa, and other epithelia.

However, the disclosure is not limited to intranasal administration and rPA pharmaceutical compositions of the disclosure may be administered by alternative means, like oral or injectable administration, as well. Useful injectable preparations include sterile suspensions, solutions, or emulsions of the active compound(s) in aqueous or oily vehicles. The compositions may also contain formulating agents, such as suspending, stabilizing, and/or dispersing agents. The formulations for injection can be presented in unit dosage form, e.g., in ampules or in multidose containers, and may contain added preservatives.

rPA compositions intended for oral use can be prepared according to any method known to the art for the manufacture of pharmaceutical compositions, and such compositions may contain one or more agents selected from the group consisting of sweetening agents, flavoring agents, coloring agents, and preserving agents in order to provide pharmaceutically elegant and palatable preparations. Tablets contain the active ingredient (including drug and/or prodrug) in admixture with non-toxic pharmaceutically acceptable excipients which are suitable for the manufacture of tablets. These excipients can be for example, inert diluents, such as calcium carbonate, sodium carbonate, lactose, calcium phosphate or sodium phosphate; granulating and disintegrating agents (e.g., corn starch or alginic acid); binding agents (e.g., starch, gelatin, or acacia); and lubricating agents (e.g., magnesium stearate, stearic acid, or talc). The tablets can be left uncoated or they can be coated by known techniques to delay disintegration and absorption in the gastrointestinal tract and thereby provide a sustained action over a longer period. For example, a time delay material such as glyceryl monostearate or glyceryl distearate can be employed. They may also be coated by the techniques described in the U.S. Pat. Nos. 4,256,108; 4,166,452; and U.S. Pat. No. 4,265,874 to form osmotic therapeutic tablets for control release. The pharmaceutical compositions of the disclosure may also be in the form of oil-in-water emulsions.

rPA liquid preparations for oral administration may take the form of, for example, elixirs, solutions, syrups, or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives, or hydrogenated edible fats); emulsifying agents (e.g., lecithin, or acacia); non-aqueous vehicles (e.g., almond oil, oily esters, ethyl alcohol, Cremophore™, or fractionated vegetable oils); and preservatives (e.g., methyl or propyl-p-hydroxybenzoates or sorbic acid). The preparations may also contain buffer salts, preservatives, flavoring, coloring, and sweetening agents as appropriate.

Exemplary dosage forms for pharmaceutical administration are described herein. Examples include but are not limited to liquids, ointments, creams, emulsions, lotions, gels, bioadhesive gels, sprays, aerosols, pastes, foams, sunscreens, capsules, microcapsules, suspensions, pessary, powder, semi-solid dosage form, etc.

The rPA pharmaceutical compositions for administration may be applied in a single administration or in multiple administrations.

The present disclosure contemplates that many variations of the described rPA compositions will be useful in the methods of the present disclosure. To determine if a candidate composition is suitable for pharmaceutical use, three criteria are analyzed. Using the methods and standards described herein, candidate rPA compositions can be easily tested to determine if they are suitable. First, the desired ingredients are prepared using the methods described herein, to determine if a buffer-stabilized rPA composition can be formed. If a buffer-stabilized rPA composition cannot be formed, the candidate is rejected. Second, the candidate buffer-stabilized rPA compositions should be stable. A buffer-stabilized rPA composition is stable if it remains in solution, with the biological activity of an rPA protein or peptide preserved for a sufficient period to allow for its intended use. For example, for pharmaceutical buffer-stabilized rPA compositions that are to be stored, shipped, etc., it may be desired that the buffer-stabilized rPA composition remain in solution form for months to years. Typical buffer-stabilized rPA compositions that are relatively unstable, will lose their form within a day. Third, the candidate pharmaceutical buffer-stabilized rPA compositions should have efficacy for its intended use. For example, the pharmaceutical buffer-stabilized rPA compositions disclosed herein should induce a protective immune response or a therapeutic effect to a detectable level.

The disclosed rPA compositions can be provided in many different types of containers and delivery systems. For example, in some embodiments of the disclosed, the rPA compositions are provided in a cream or other solid or semi-solid form. The disclosed rPA compositions may be incorporated into hydrogel formulations.

The rPA compositions can be delivered (e.g., to a subject or customers) in any suitable container. Suitable containers can be used that provide one or more single use or multi-use dosages of the rPA compositions for the desired application. In some embodiments of the disclosed, the rPA compositions are provided in a suspension or liquid form. Such rPA compositions can be delivered in any suitable container including spray bottles and any suitable pressurized spray device. Such spray bottles may be suitable for delivering the compositions intranasally or via inhalation. These containers can further be packaged with instructions for use to form kits.

### V. Definitions

As used herein, “about” will be understood by persons of ordinary skill in the art and will vary to some extent depending upon the context in which it is used. If there are uses of the term which are not clear to persons of ordinary skill in the art given the context in which it is used, “about” will mean up to plus or minus 10% of the particular term.

As used herein, the term “adjuvant” refers to an agent that increases the immune response to an antigen (e.g., a pathogen).

As used herein, the term “immune response” refers to a subject's (e.g., a human or another animal) response by the immune system to immunogens (i.e., antigens) which the subject's immune system recognizes as foreign. Immune responses include both cell-mediated immune responses (responses mediated by antigen-specific T cells and non-specific cells of the immune system) and humoral immune responses (responses mediated by antibodies present in the plasma lymph, and tissue fluids). The term “immune response” encompasses both the initial responses to an immunogen (e.g., a pathogen) as well as memory responses that are a result of “acquired immunity.”

The terms “chelator” or “chelating agent” refer to any materials having more than one atom with a lone pair of electrons that are available to bond to a metal ion.

As used herein, the term “enhanced immunity” refers to an increase in the level of acquired immunity to a given pathogen following administration of a vaccine of the present disclosure relative to the level of acquired immunity when a vaccine of the present disclosure has not been administered.

As used herein, the term “immunogen” refers to an antigen that is capable of eliciting an immune response in a subject. In preferred embodiments, immunogens elicit immunity against the immunogen (e.g., a pathogen or a pathogen product) when administered in combination with a nanoemulsion of the present disclosure.

As used herein, the term “intranasal(ly)” refers to application of the compositions of the present disclosure to the surface of the skin and mucosal cells and tissues of the nasal passages, e.g., nasal mucosa, sinus cavity, nasal turbinates, or other tissues and cells which line the nasal passages.

The term “nanoemulsion,” as used herein, includes small oil-in-water dispersions or droplets, as well as other lipid structures which can form as a result of hydrophobic forces which drive apolar residues (i.e., long hydrocarbon chains) away from water and drive polar head groups toward water, when a water immiscible oily phase is mixed with an aqueous phase. These other lipid structures include, but are not limited to, unilamellar, paucilamellar, and multilamellar lipid vesicles, micelles, and lamellar phases. The present disclosure contemplates that one skilled in the art will appreciate this distinction when necessary for understanding the specific embodiments herein disclosed.

The terms “pharmaceutically acceptable” or “pharmacologically acceptable,” as used herein, refer to compositions that do not substantially produce adverse allergic or adverse immunological reactions when administered to a host (e.g., an animal or a human). Such formulations include any pharmaceutically acceptable dosage form. Examples of such pharmaceutically acceptable dosage forms include, but are not limited to, dips, sprays, seed dressings, stem injections, lyophilized dosage forms, sprays, and mists. As used herein, “pharmaceutically acceptable carrier” includes any and all solvents, dispersion media, coatings, wetting agents (e.g., sodium lauryl sulfate), isotonic and absorption delaying agents, disintegrants (e.g., potato starch or sodium starch glycolate), and the like.

As used herein, the term “topical(ly)” refers to application of the compositions of the present disclosure to the surface of the skin and mucosal cells and tissues (e.g., buccal, lingual, sublingual, masticatory, respiratory or nasal mucosa, nasal turbinates and other tissues and cells which line hollow organs or body cavities).

As used herein, “viral particles” refers to mature virions, partial virions, empty capsids, defective interfering particles, and viral envelopes.

“Administration” can be effected in one dose, continuously or intermittently throughout the course of treatment. Methods of determining the most effective means and dosage of administration are known to those of skill in the art and will vary with the composition used for therapy, the purpose of the therapy, the target cell being treated, the disease being treated and the subject being treated. Single or multiple administrations can be carried out with the dose level and pattern being selected by the treating physician. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated, and target cell or tissue. Non-limiting examples of route of administration include oral administration, nasal administration, inhalation, injection, and topical application.

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Embodiments defined by each of these transition terms are within the scope of this disclosure. Accordingly, it is intended that the methods and compositions can include additional steps and components (comprising) or alternatively including steps and compositions of no significance (consisting essentially of) or alternatively, intending only the stated method steps or compositions (consisting of).

The disclosed is further described by reference to the following examples, which are provided for illustration only. The disclosed is not limited to the examples, but rather includes all variations that are evident from the teachings provided herein. All publicly available documents referenced herein, including but not limited to U.S. patents, are specifically incorporated by reference.

### EXAMPLES

### Example 1

### Stabilization of rPA

The purpose of this example was to try to optimize various compositions to stabilize the secondary and tertiary structures of rPA protein by proactively screening and addressing all of the destabilizing or un-stabilizing factors that would affect the structure and lead to aggregation and degradation of the rPA protein.

Selection of Stabilizing Excipients for Vaccine Formulation: A screening study was performed on various formulations shown in the table below. These are screening stability studies that were used to guide formulation development and narrow in on the excipient to be used in the final formulation selection. Various prototype formulations were placed on informal stability studies

Table 1 describes the various buffer systems and additional stabilizing excipient that were investigated. Various prototype formulations were placed on informal stability studies and are described in the tables below. In particular, the different buffer systems, either phosphate or TRIS buffer, were evaluated as the base and additional excipients were then added in a matrix type design.

TABLE 1
Stabilizing excipients and function

| Excipients/Systems | Example of Excipients | Function |
|---|---|---|
| Buffer Systems | 10 mM PBS buffer (pH 7.4,) | Control the pH of the system; Optimized |
| | 10, 80 mM TRIS buffer | solubility based on the Isoelectric Point (pI) |
| | (pH 8.0) | of the rPA Protein; Buffering components to |
| | | control pH (effects the pI) |
| Salts | 100-150 mM Sodium | Increase the surface tension of water ionic |
| | Chloride | strength. |
| | | Optimize Ionic strength; if there is calcium |
| | | dependent folding of the rPA protein domain |
| Sugars | 5, 15% Trehalose | Protect rPA protein native conformation, |
| | 5% Sucrose | alters tonicity and osmolality |
| Amino Acids | 20, 60 mM Histidine | Direct rPA protein binding, buffering |
| | | capacity, and antioxidant properties, |
| | | suppressing the aggregation of folding |
| | | intermediates, radical attacks by reactive |
| | | oxygen and nitrogen species, prevents |
| | | denaturation of amino acids. |
| Storage: | Nitrogen, Argon | Hydrogen bonds are broken by increased |
| Inert Gas, | Glass covered by Foil | translational energy, shearing of hydrogen |
| Limit Head Space, | (Amber glass may have | bonds, |
| Protect from Light, | leachables) | Inclusion of inert gas to prevent oxidation |
| Low Agitation | Fill Volume | Protection from light |
| | No vortexing, Simple mixing | |
| | with low shear. | |

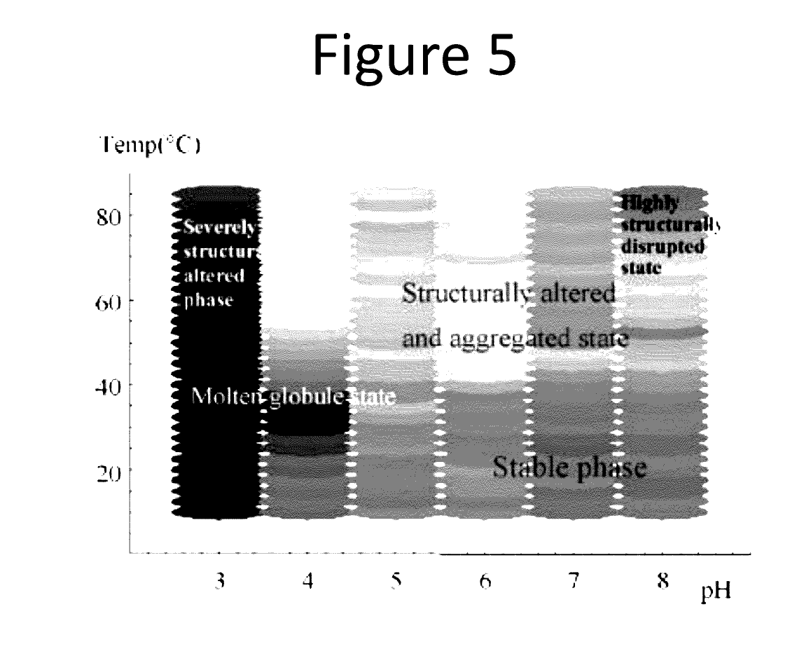

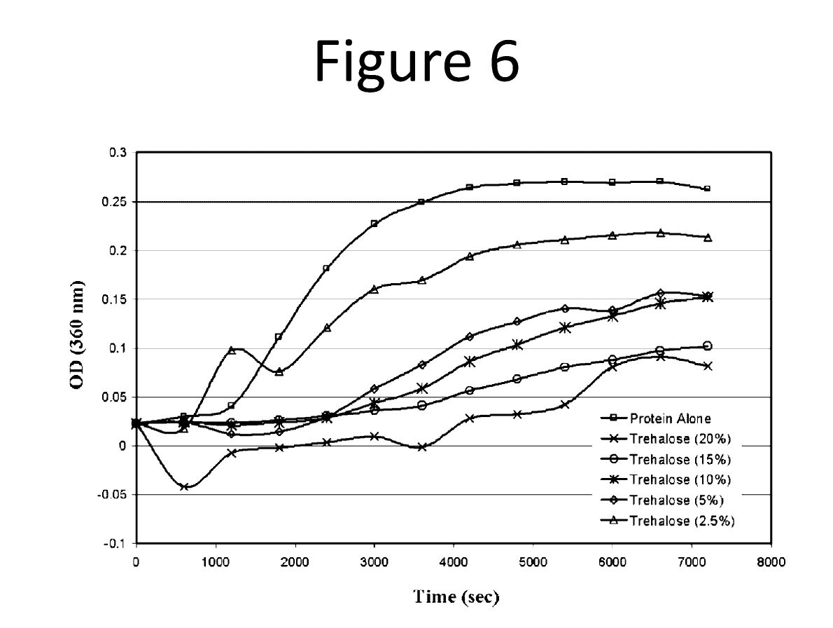

The selection of a stabilizing sugar helps protect the protein antigen rPA at higher temperatures. FIG. 5 shows the best pH and FIG. 6 shows the optimal concentration of trehalose to protect the rPA protein antigen from aggregation. Jiang et al., “Anthrax Vaccine Powder Formulations for Nasal Mucosal Delivery,” Journal of Pharmaceutical Sciences, 95: 80-96 (2006).

The effect of pH and temperature was evaluated via a phase diagram, and the most stable phase was found to in the lower right-hand corner of FIG. 5, where the pH was from 7-8. Below this pH, molten globule-like state structures are apparent around pH 3. Thus, pH 7.4-8 was the targeted pH for the rPA protein antigen formulations.

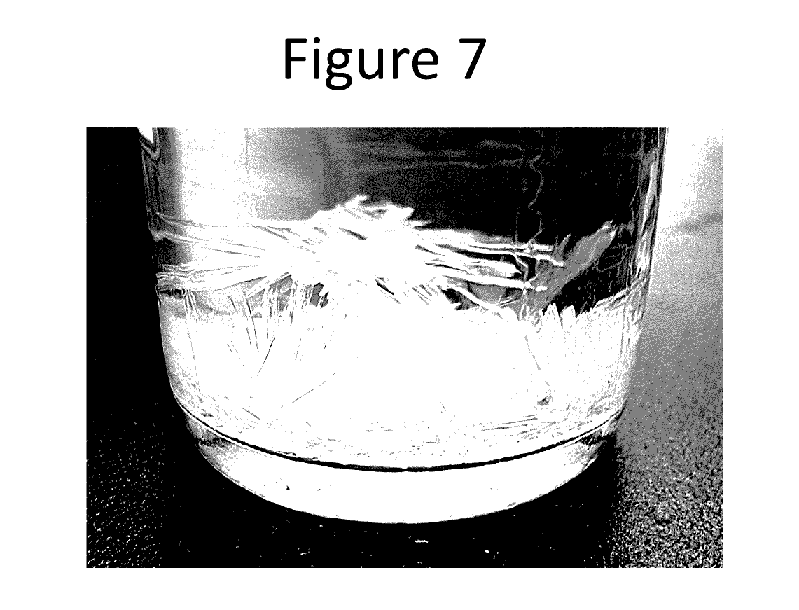

A potential stabilizer, trehalose, is also identified in Jiang et al., as several concentrations of rPA protein antigen formulations comprising trehelose were evaluated while heating an rPA solution. The disaccharide trehalose was found to be one of the most effective aggregation inhibitors. The extent of inhibition of rPA aggregation was concentration-dependent, as shown in FIG. 6. In this case, about 5% or higher concentrations of trehalose elicited 50% inhibition of rPA protein aggregation, consisting of a mixture of secondary structure moieties (e.g., a-helix and b-sheet). Thus, 5% and 15% trehalose were the two concentrations further investigated regarding promotion of rPA protein antigen stability. Sucrose and mannitol were selected for further study. However, following this selection it was discovered that mannitol crystalized out of solution on prolonged storage at 2-8° C., as shown in FIG. 7. Hence, mannitol was removed from further formulation consideration.

### Example 2

### Prototype Formulation Containing rPA

The purpose of this example was to identify a rPA prototype formulation design for stability of an rPA protein antigen. The tested formulations comprised an rPA antigen in a stabilized buffer system. Exemplary formulations of the stabilizing system are show in Tables 5-7.

The rPA concentrations used in the studies bracketed at concentrations of 100 μg rPA/mL and 500 μg rPA/mL rPA. The base formulation in a phosphate buffer system was placed on stability at 5° C., 25° C. and 40° C. for 1 and 3 months. The rPA prototype formulations were stored at −20° C., 5° C. and 25° C. for longer stability time points (e.g. 1, 3, 6 and up to 12 months). The rPA prototype formulations were also stored at 40° C. and were analyzed at 1, 3 and 6 months.

The stability assays included physical appearance, pH, particle size, cetylpyridinium chloride potency (CPC potency, % CPC), qualitative Western Blot for rPA (MW=83 kDa), rPA potency (% rPA) was determined by RP-HPLC and SEC-HPLC. CPC is a compound present in the nanoemulsions, and the measurement of CPC can be used as a “marker” to determine if the potency of the nanoemulsion adjuvant decreases over time.

FIGS. 2-4 show schematic diagrams of the decision trees used in the selection of the stabilizing excipients in the methods of the invention. The three series of rPA prototype formulations and the excipient variable that were optimized are highlighted in the figures.

### Example 3

### Effect of Excipients on the Thermostability of rPA

Various systems were tested to confirm that the disclosed compositions and methods could stabilize and preserve rPA in a solution. Table 2 describes the various buffer systems and additional stabilizing excipients that were investigated. These are heat screening stability studies that were used to guide formulation development and narrow in on the excipients. Various prototype formulations were placed on informal stability and are described, as shown in Table 4.

TABLE 2
Stabilizing Excipients and Function.

| Excipients/Systems | Example of Excipients | Function |
|---|---|---|
| Buffer Systems | 10 mM PBS buffer (pH 7.4) | Control the pH of the system; Optimize |
| | 10, 80 mM TRIS buffer | solubility based on the Isoelectric Point |
| | (pH 8.0) | (pI) of the Protein (rPA pI = 5.6); |
| | | Buffering components to control pH |
| | | (affects the pI) |
| Salts | 50-150 mM Sodium | Increase the surface tension of water |
| | Chloride | ionic strength. |
| | 50-150 mM Calcium | Optimize Ionic strength; if there is |
| | Chloride | calcium dependent folding of the rPA |
| | | protein domain |
| Sugars | 5-15% Trehalose | Protect rPA protein native conformation, |
| | 5, 10% Sucrose | alters tonicity and osmolality |
| | 5, 10% Glycerol | |
| | 5-10% Mannitol | |
| Amino Acids | 20-60 mM Histidine | Direct rPA protein binding, buffering |
| | 16 mM Glutathione | capacity and antioxidant properties, |
| | 10 mM Alanine | suppressing the aggregation of folding |
| | | intermediates, radical attacks by reactive |
| | | oxygen and nitrogen species, prevents |
| | | denaturation of amino acids |
| Storage: | Nitrogen, Argon | Hydrogen bonds are broken by increased |
| Inert Gas, | Glass covered by Foil | translational energy, shearing of |
| Limit Head Space, | (Amber glass may have | hydrogen bonds, |
| Protect from Light, | leachables) | Inclusion of inert gas to prevent |
| Low Agitation | Fill Volume | oxidation |
| | No vortexing, simple | Protection from light |
| | mixing with low shear. | |

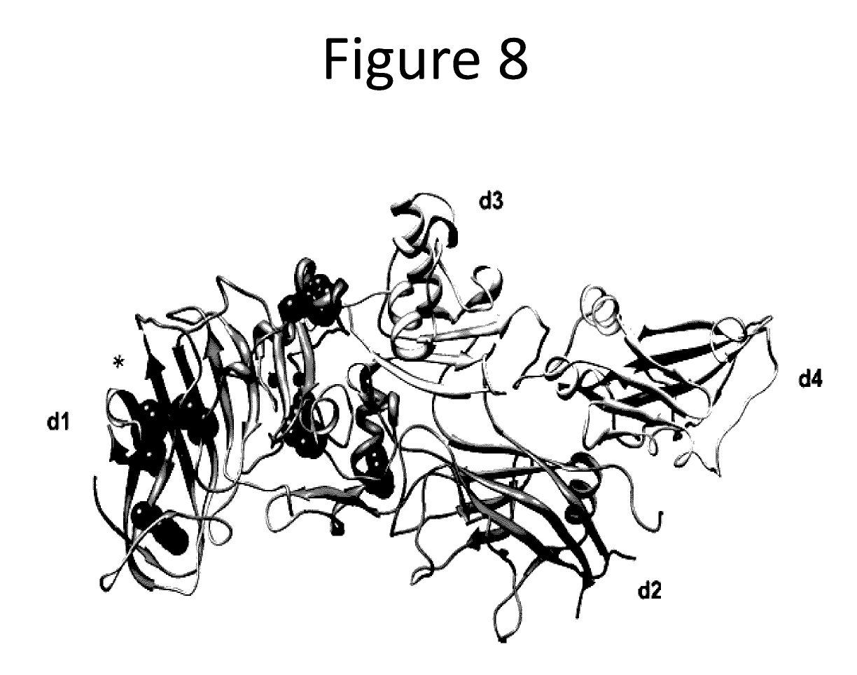

The selection of the buffer used to formulate rPA protein was shown to have a great effect on the stability of rPA protein. It is also understood that the pH of phosphate buffer solutions can change significantly at low temperatures, and this has been ascribed to enthalpic effects on the proton equilibrium as well as selective precipitation of buffer components upon cooling. If left unaccounted for, these pH changes could lead to damage to the rPA protein structure upon storage at low temperatures. Also, phosphates sequester divalent cations, such as Ca²⁺ and Mg²⁺. This may be problematic for rPA in longer-term storage due to calcium molecules located in the domain d1 of the rPA protein structure as shown in FIG. 8.

TRIS is a buffer used to maintain the pH within a relatively narrow range. TRIS has a slightly alkaline buffering capacity in the 7-9.2 pH range. TRIS has a pKₐ of 8.06 at 25° C. It has a low salt effect, no interference from isotonic saline solution, and minimal concentration impact on the dissociation constant. It will not bind calcium or magnesium cations, avoiding this type of interference or precipitation. It is chemically stable, both alone and in aqueous solution, so storage of stock solutions is convenient. It has insignificant light absorbance characteristics between 240 nm and 700 nm, so its use will not interfere in colorimetric measurements. It has acceptable toxicity properties, and is widely used in pharmaceutical applications. Thus, phosphate and TRIS buffered systems were investigated.

The isoelectric point, sometimes abbreviated to pI, is the pH at which a particular molecule or surface carries no net electrical charge. The pI value can affect the solubility of a molecule at a given pH. Amino acids that make up proteins may be positive, negative, neutral, or polar in nature, and together give a protein its overall charge. At a pH below their pI, proteins carry a net positive charge; above their pI they carry a net negative charge. The larger the difference between the pI and the pH, the greater net charge is on the protein. The pI of rPA is 5.6. Hence, two pH units above the pI (e.g. 5.6 to 7.6) is theoretically the best pH for rPA based on its pI, unless other studies are performed to optimize the pH with other excipients (e.g. see trehalose discussion below). Thus, pH 7.4-8 was the targeted pH range for the prototype rPA formulations. The disaccharide trehalose was found to be the most effective aggregation inhibitor. Thus, 5% and 15% trehalose were the two concentrations that was investigated. Sucrose was also evaluated.

rPA protein is susceptible to oxidative damage through reaction of certain amino acids with oxygen radicals present in their environment. Methionine, cysteine, histidine, tryptophan, and tyrosine are susceptible to oxidation. Oxidation can alter rPA protein physical chemical characteristics (e.g. folding) and lead to aggregation or fragmentation. In particular, histidine residues are highly sensitive to oxidation through reaction with the imidazole rings. Controlling or enhancing factors, such as pH, temperature, light exposure, and buffer composition will mitigate the effects of oxidation. The addition of freely soluble amino acids, such as histidine, will help protect the native conformational protein structure of rPA by acting as a surrogate for the oxidative chemical species that promote oxidiation of the intact rPA protein. These free amino acids in effect act as an effective anitoxidant. For rPA protein, there are a high percentage of histidine residues in the structure that need to be protected from oxidation. Thus, histidine alone and in combination with other amino acids were investigated with respect to improving the thermo-labile stability of rPA.

### Example 4

### Heat Screening Study of rPA

The heat screening study focused on testing formulations containing two buffers (PBS or TRIS) and excipients, such as sodium chloride (NaCl), sucrose, histidine, and glycerol. The rPA aqueous solutions tested are listed in Table 4. The concentration of rPA was 500 μg/mL.

The following is the procedure and acceptance criteria for the rPA aqueous solution plus excipients screening experiments:
1) Prepare desired rPA buffer formulations (control and test formulations)
2) Heat test formulation in heating block set at 49° C. for 5 minutes.
3) Assess percent area of rPA peak following incubation versus control.
4) Select the buffer formulations that have >70% area and no secondary peak at 15 minutes as assessed by SEC.

### Example 5

### Development of rPA SEC and RP-HPLC Method

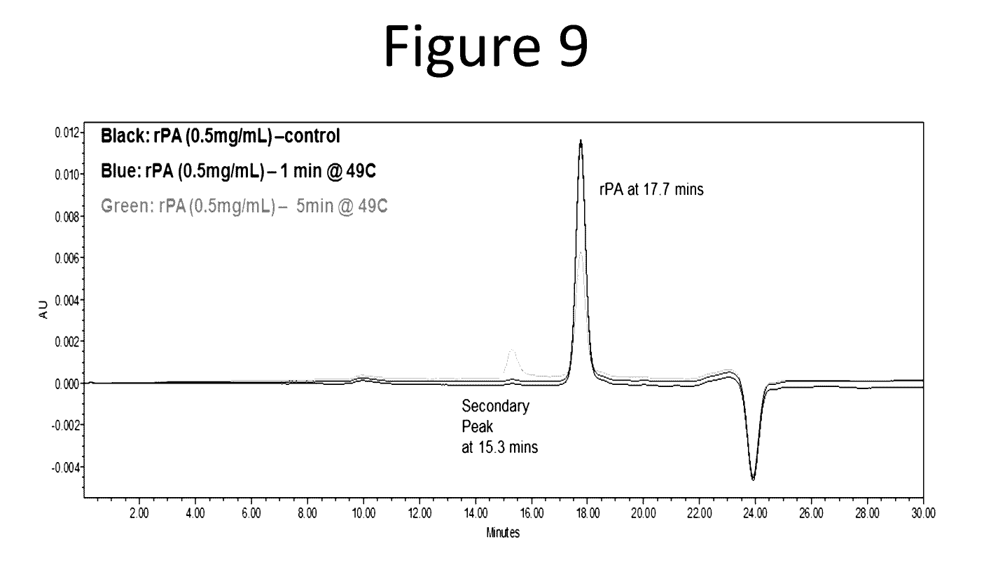

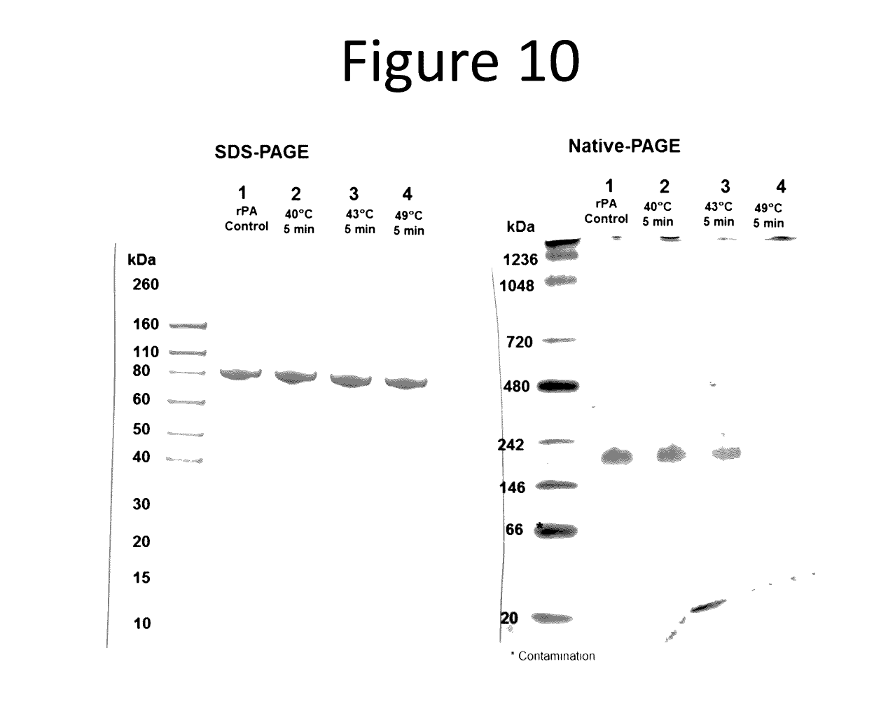

Incubation of the rPA solution at 49° C. for 5 minutes using a heating block caused thermal aggregation of rPA (Table 3 and FIG. 9); whereas at the other conditions the rPA was stable. Thermal aggregation at this condition was also confirmed with native PAGE (FIG. 10). Thus, 49° C. for 5 minutes was the condition selected to rapidly screen various rPA formulations shown in Table 4.

The screening method for the stabilizing excipients consisted of using size exclusion chromatography (SEC-HPLC) to compare the area of the rPA peak in different rPA formulations heated to 49° C. for 5 minutes compared to a non-heated sample. Formulations that had a greater than 80% peak area and no secondary peak at 15 minutes on SEC-HPLC were selected was considered stable.

TABLE 3
Effect of Temperature and Time on rPA
Physical Stability using SEC-HPLC.

| Screening (Heating) Condition | % rPA Area SEC-HPLC |
|---|---|
| Control (No heating) | 100.0 |
| 1 min at 40° C. | 108.4 |
| 5 min at 40° C. | 104.2 |
| 1 min at 43° C. | 104.2 |
| 5 min at 43° C. | 104.0 |
| 1 min at 49° C. | 103.8 |
| 5 min at 49° C. | 37.9 |

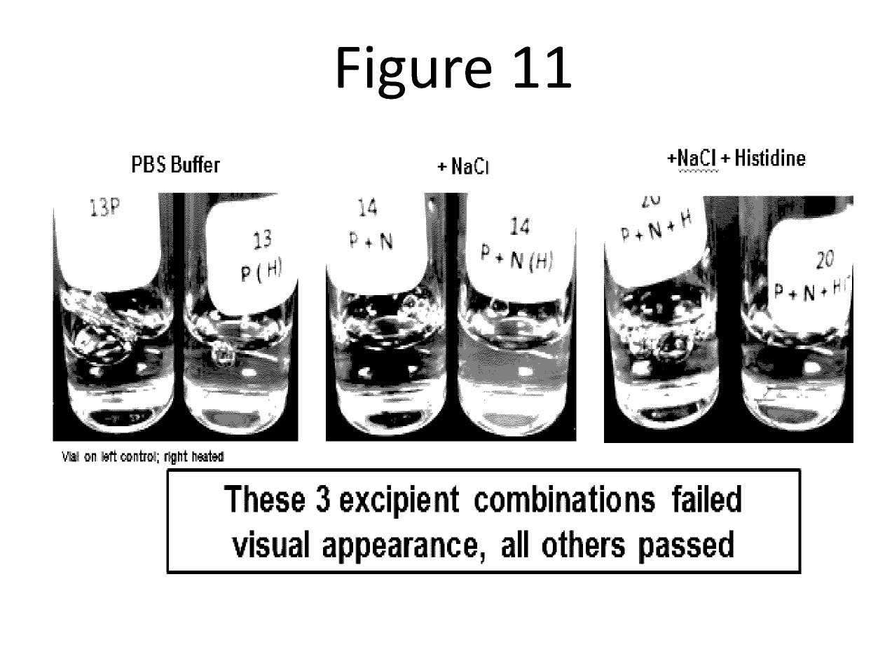

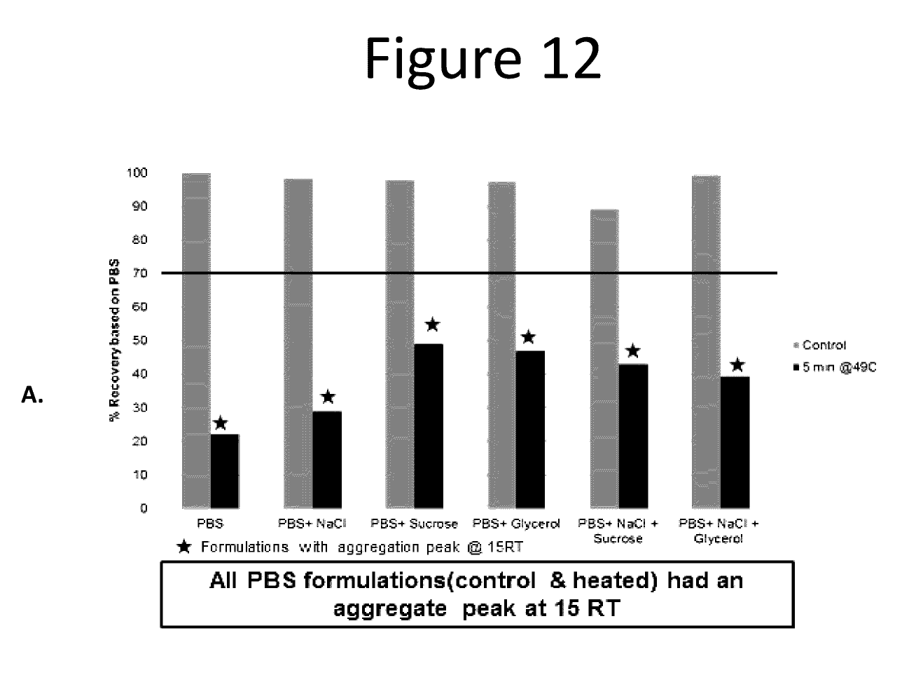

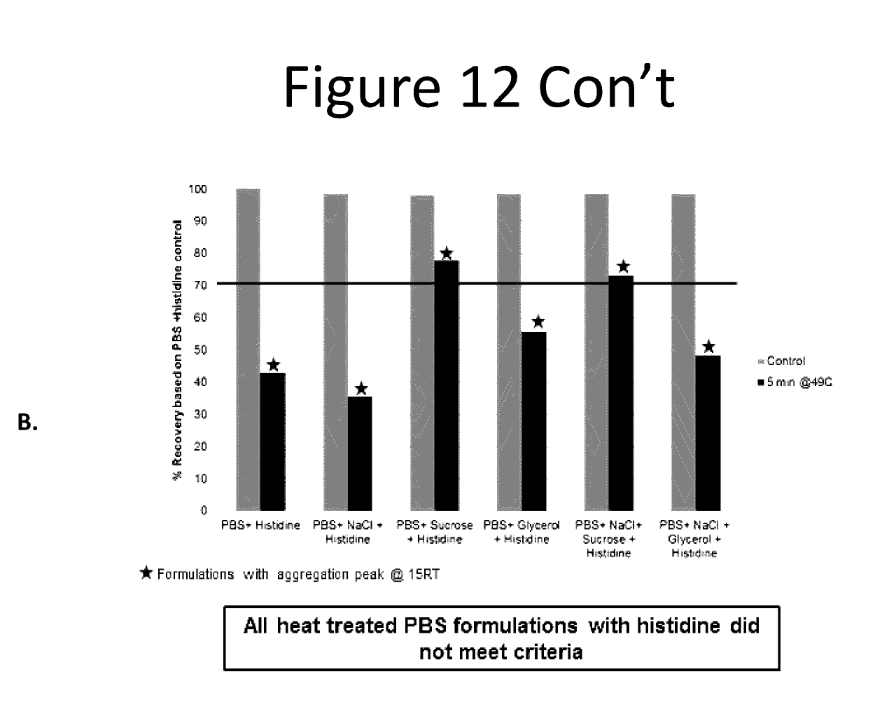

FIG. 11 shows that when the sodium phosphate system was heated, the solutions turned turbid. When the solution turns turbid, this indicates aggregation and precipitation of the rPA protein. These three compositions in FIG. 11 clearly failed visual appearance. FIG. 12 shows that all the formulations tested with sodium phosphate and additional excipients when heated lost rPA recovery. All the formulations, except two, were well below the 70% cut off point. The two formulations above 70%, however, showed a 15 minute retention time rPA aggregation peak, as indicated by a star.

TABLE 4
List of Excipient used in rPA Aqueous Screening Studies.

| | 10 mM | |
|---|---|---|
| | Sodium | 10 mM |
| | Phosphate | TRIS |
| Excipients | (pH 7.4) | (pH 8) |
| Control | X | X |
| 50 mM NaCl | X | X |
| 5% Sucrose | X | X |
| 20 mM Histidine | X | X |
| 5% Glycerol | X | X |
| 50 mM NaCl + 5% Sucrose | X | X |
| 50 mM NaCl + 5% Glycerol | X | X |
| 50 mM NaCl + 20 mM Histidine | X | X |
| 5% sucrose + 20 mM Histidine | X | X |
| 5% Glycerol + 20 mM Histidine | X | X |
| 20 mM Histidine + 50 mM NaCl + 5% Sucrose | X | X |
| 20 mM Histidine + 50 mM NaCl + 5% Glycerol | X | X |

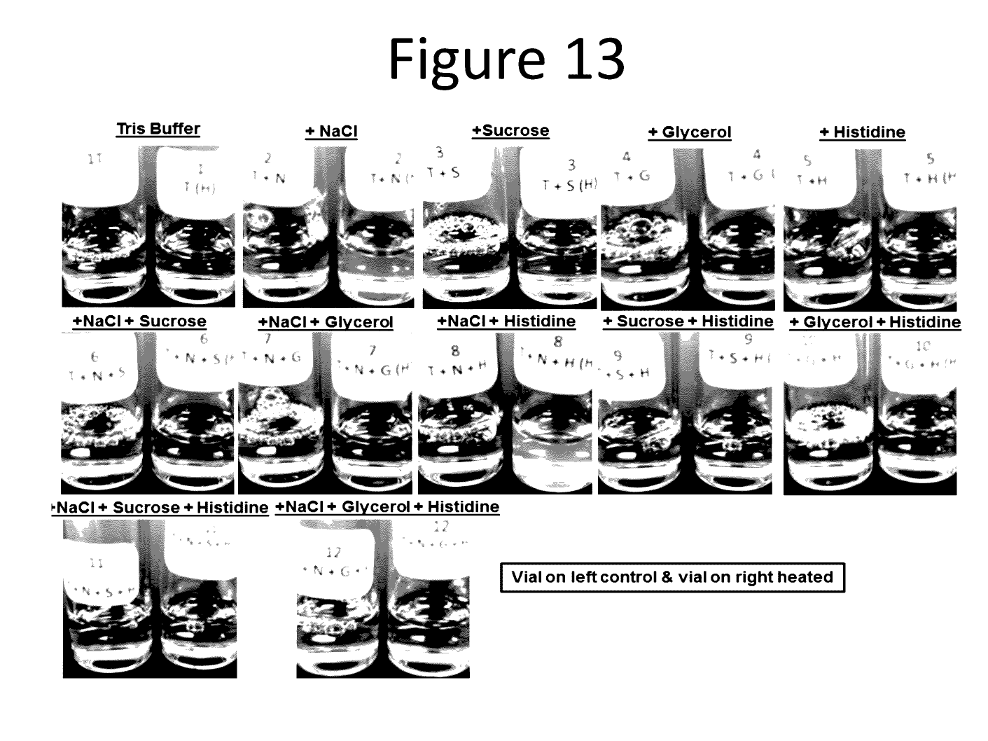

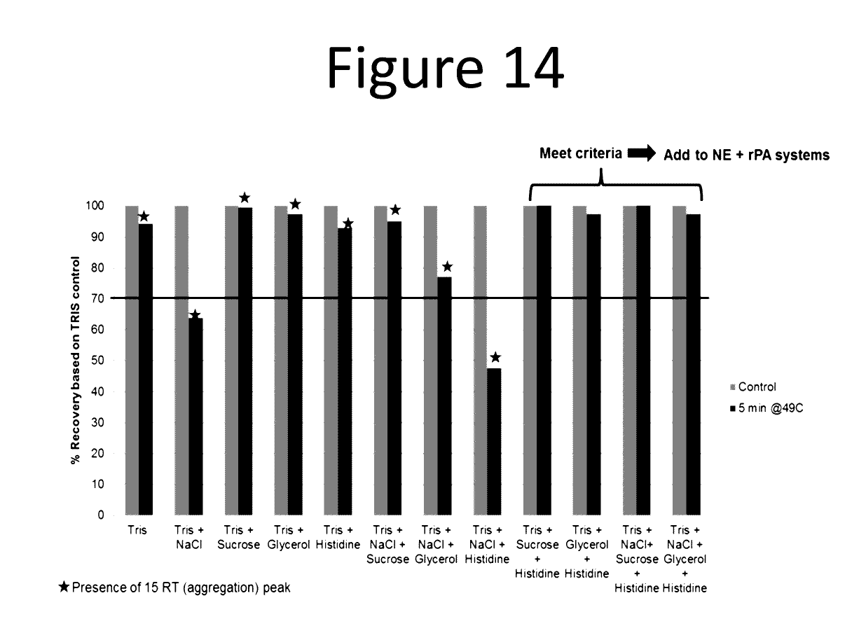

FIG. 13 shows the physical appearance of the TRIS systems with various excipients before and after heating. A couple of turbid solutions (+NaCl, +NaCl+Histidine) developed after heating, which indicates aggregation and precipitation of the rPA protein. FIG. 14 show that four compositions met the acceptance criteria.

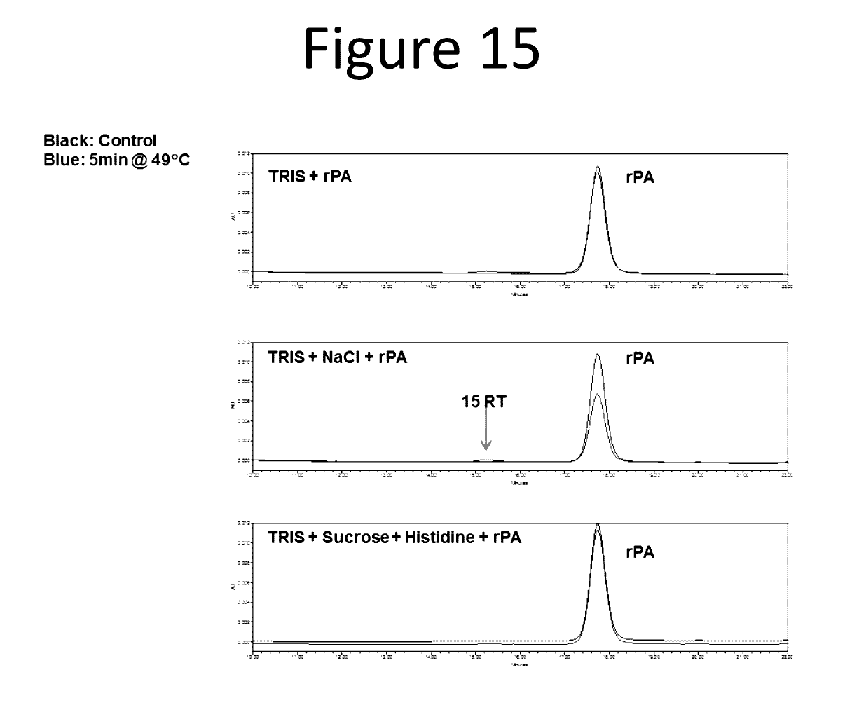

In summary, the screening method indicated that the TRIS buffer system, rather than phosphate buffer system was the better buffer with respect to rPA stability (FIGS. 13 and 14). None of the rPA PBS solutions listed in the table above met the acceptance criteria. The recovery of rPA for all the samples following heating was less than 70%. Only two of these solutions, the histidine, and sucrose with or without NaCl had recovery of rPA greater than 70%. All other formulations had percent rPA recovery less than 60%. Additionally, for all of these formulations the unheated control and the formulations heated for 5 minutes at 49° C. exhibited an aggregate peak at a retention time of 15 minutes as determined by SEC-HPLC. FIG. 15 shows some example chromatographs. Four of the heat-treated TRIS buffer formulations met the acceptance criteria as indicated in FIG. 15.

### Example 6

### Effect of Excipients on the Long-Term Stability of rP (Prototype Formulations)

The rPA concentrations used bracketed at 100 μg rPA/ml and 500 μg rPA/mL. The rPA prototype formulations were stored at −20° C., +5° C. and +25° C., and stability of the different formulations was determined after 1, 3, 6, 9, and 12 months. Formulations of rPA stabilized solutions were also stored at 40° C. and analyzed at 1, 3, and 6 months. The stability assays are listed in Appendix 1, 2 and 3 and include: physical appearance, pH, particle size, qualitative Western Blots for rPA, rPA determined by RP-HPLC and SEC-HPLC. The Western blots method for rPA and were probed using the Novus rabbit polyclonal whole sera antibody as the primary antibody.

FIGS. 2-4 show schematics of the decision trees used in the selection of the stabilizing excipients. Between each prototype there was an additional screening step to optimize at least one of the excipients (i.e. the buffer in prototype 1/FIG. 2; Trehalose is prototype 2/FIG. 3; and Glutathione in Prototype 3/FIG. 4).

Tables 5-7 list the formulations for Prototypes 1, 2 and 3 placed on informal stability at −20° C., 5° C., 25° C., and 40° C. at various time points.

TABLE 5
Composition of rPA Prototype 1 Formulations.

| | | rPA Prototype 1 Excipient Compositions | | | | | |
|---|---|---|---|---|---|---|---|
| | | rPA | % | Buffer | NaCl | Histidine | Sucrose |
| Lot # | Type | (μg/mL) | NE | System | (mM) | (mM) | (mM) |
| X-1596 | rPA aqueous | 100 | 0 | 10 mM | 100 | 20 | 5 |
| | | | | PBS | | | |
| X-1595 | rPA aqueous | 500 | 0 | 10 mM | 100 | 20 | 5 |
| | | | | PBS | | | |
| X-1601 | rPA aqueous | 100 | 0 | 10 mM | 150 | 20 | 5 |
| | | | | TRIS | | | |
| X-1600 | rPA aqueous | 500 | 0 | 10 mM | 150 | 20 | 5 |
| | | | | TRIS | | | |

TABLE 6
Composition of rPA Prototype 2 Formulations.

| | | rPA Prototype 2 Excipient Compositions | | | | | | | |
|---|---|---|---|---|---|---|---|---|---|
| | | rPA | % | Buffer | NaCl | Histidine | Trehalose | Glutathione | EDTA |
| Lot # | Type | (μg/mL) | NE | System | (mM) | (mM) | (%) | (mM) | (mM) |
| X-1624 | rPA | 100 | 0 | 80 mM | 150 | 20 | 5 | 16 | 0.5 |
| | aqueous | | | TRIS | | | | | |
| X-1626 | rPA | 500 | 0 | 80 mM | 150 | 20 | 5 | 16 | 0.5 |
| | aqueous | | | TRIS | | | | | |
| X-1629 | rPA | 100 | 0 | 80 mM | 150 | 20 | 15 | 16 | 0.5 |
| | aqueous | | | TRIS | | | | | |
| X-1631 | rPA | 500 | 0 | 80 mM | 150 | 20 | 15 | 16 | 0.5 |
| | aqueous | | | TRIS | | | | | |

Table 7
Composition of rPA Prototype 3 Formulations.

| | | rPA Prototype 2 Excipient Compositions | | | | | | |
|---|---|---|---|---|---|---|---|---|
| | | rPA | % | Buffer | NaCl | Histidine | Trehalose | Glutathione |
| Lot # | Type | (μg/mL) | NE | System | (mM) | (mM) | (%) | (mM) |
| X-1634 | rPA | 100 | 0 | 80 mM TRIS | 150 | 60 | 15 | 0 |
| | aqueous | | | | | | | |
| X-1636 | rPA | 500 | 0 | 80 mM TRIS | 150 | 60 | 15 | 0 |
| | aqueous | | | | | | | |
| X-1639 | rPA | 100 | 0 | 80 mM TRIS | 150 | 60 | 15 | 16 |
| | aqueous | | | | | | | |
| X-1641 | rPA | 500 | 0 | 80 mM TRIS | 150 | 60 | 15 | 16 |
| | aqueous | | | | | | | |

Various rPA formulations were filled into 1.8 mL, Type 1 glass, vials with a PTFE-lined screw cap. The stability parameters assessed for these formulations were physical appearance, pH, mean particle size, non-quantitative rPA Western blot, and rPA by RP-HPLC and SEC-HPLC as described in Table 8. Dynamic light scattering using the Malvern Zetasizer was used to determine particle size, particle size distribution profiles, and polydispersity index.

A number of stability indicating analytical methods were developed for analysis of the screening formulations and prototypes. Table 8 shows the methods that were developed and the acceptance criteria for each method.

TABLE 8
Test Method and Acceptance Criteria for the
rPA Formulations Placed on Informal Stability

| | | Acceptance Criteria for |
|---|---|---|
| | | Each rPA Formulation Type |
| Stability | Test | rPA Buffered Solution |
| Parameter | Method | (rPA Aqueous) |
| Physical | Visual | No Precipitation and/or |
| Appearance | | Cloudy Solution |
| pH | pH Meter | ±0.5 |
| Particle Size | Dynamic | Peak |
| | Light | 8-20 nm |
| | Scattering | |
| PdI | Dynamic | — |
| | Light | |
| | Scattering | |
| 83 kD Band | Western | Band Present |
| | Blot | |
| rPA | SEC-HPLC | ≧80% |
| % Label Claim* | RP-HPLC | |

*The % rPA label claim is used to describe the % rPA recovered.

### Example 7

### Physical Appearance Test Method

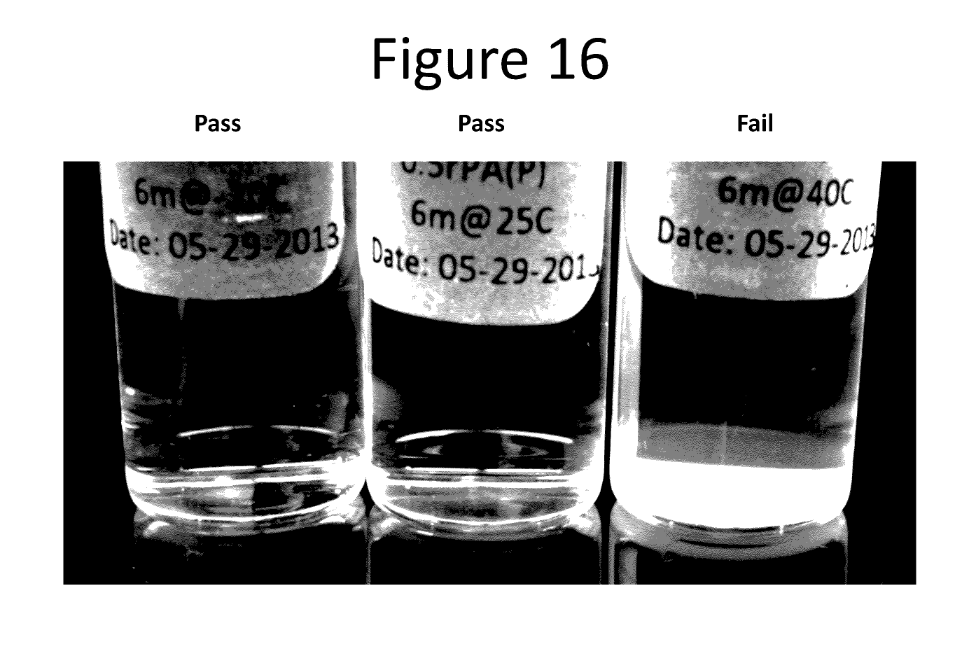

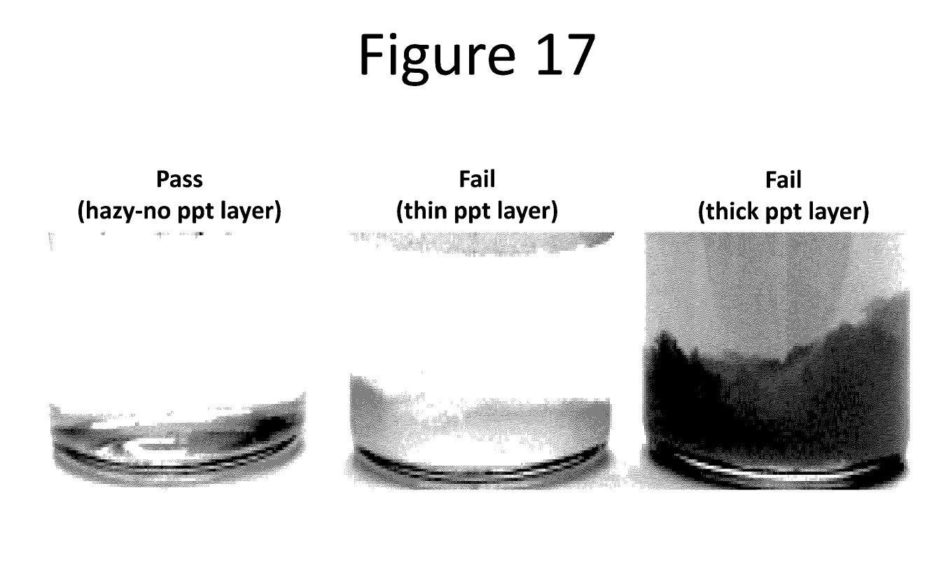

Physical appearance of the rPA solution formulations was determined at the initial time point and at different time points under various storage conditions. The physical appearance observation was then recorded and evaluated using the acceptance criteria in Table 9. FIGS. 16 and 17 illustrate examples of the acceptance criteria.

TABLE 9
Stability Parameters, Description, and Acceptance Criteria

| Stability | | | |
|---|---|---|---|
| Parameter | Description | Acceptance Criteria | |
| Precipitate | Precipitation (ppt) of rPA. | Pass: | Fail: |
| (ppt) | Remixing will not restore | None | Thin/Moderate |
| | homogeneity. | Hazy | precipitation layer |
| | | appearance, | Thick/Extreme |
| | | no ppt layer | precipitation layer |
| | | Mil | |

### Example 8

### pH Assessment

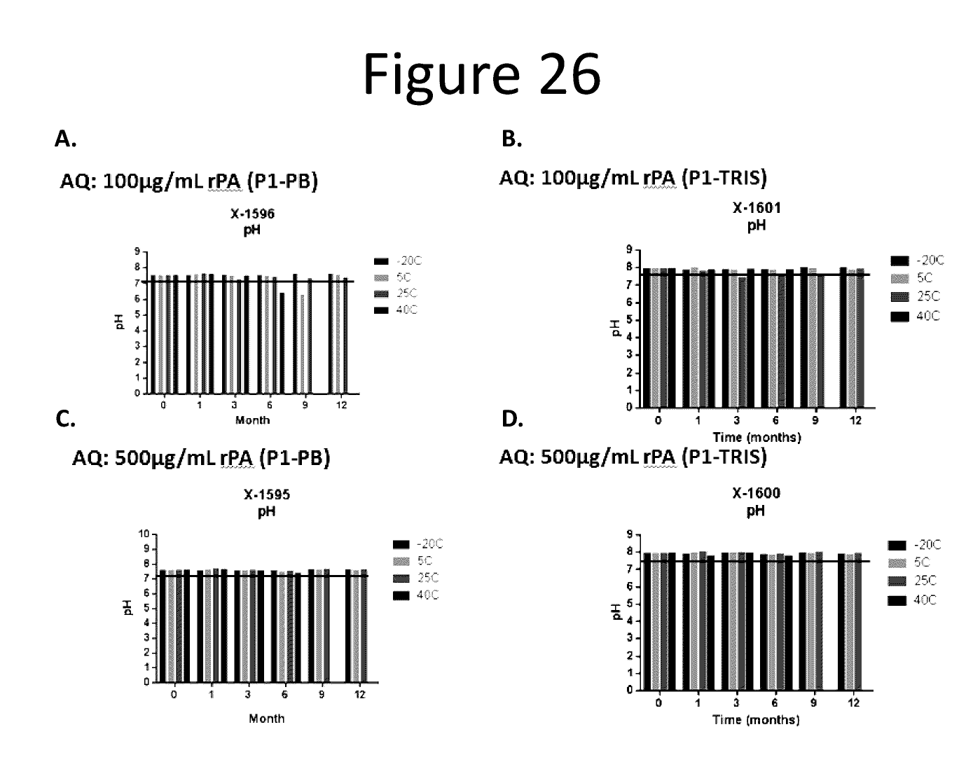

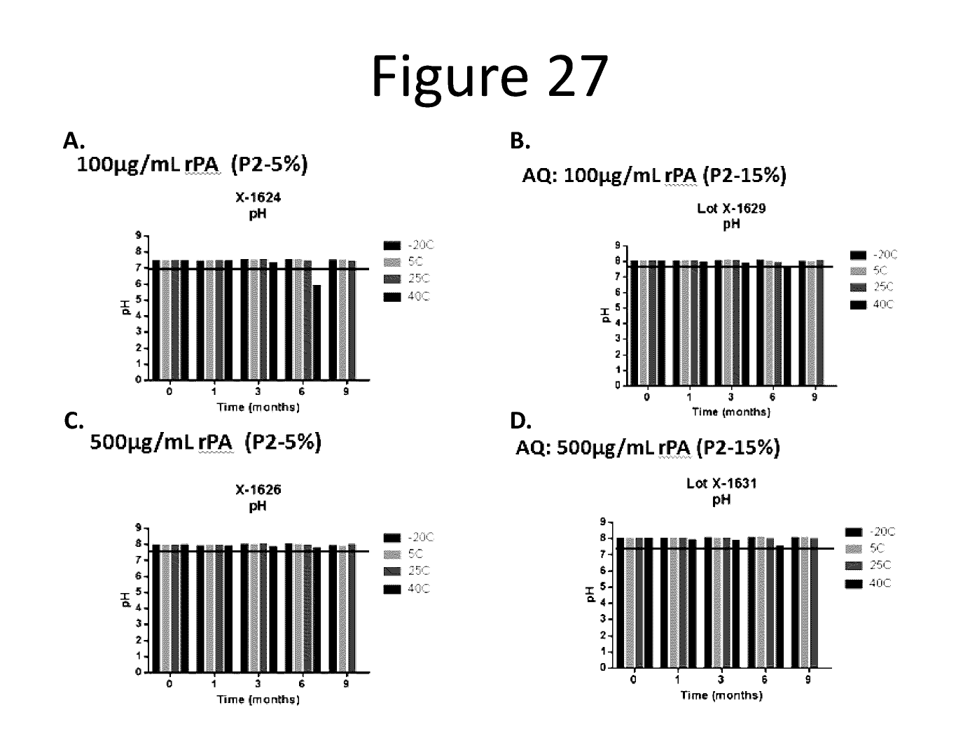

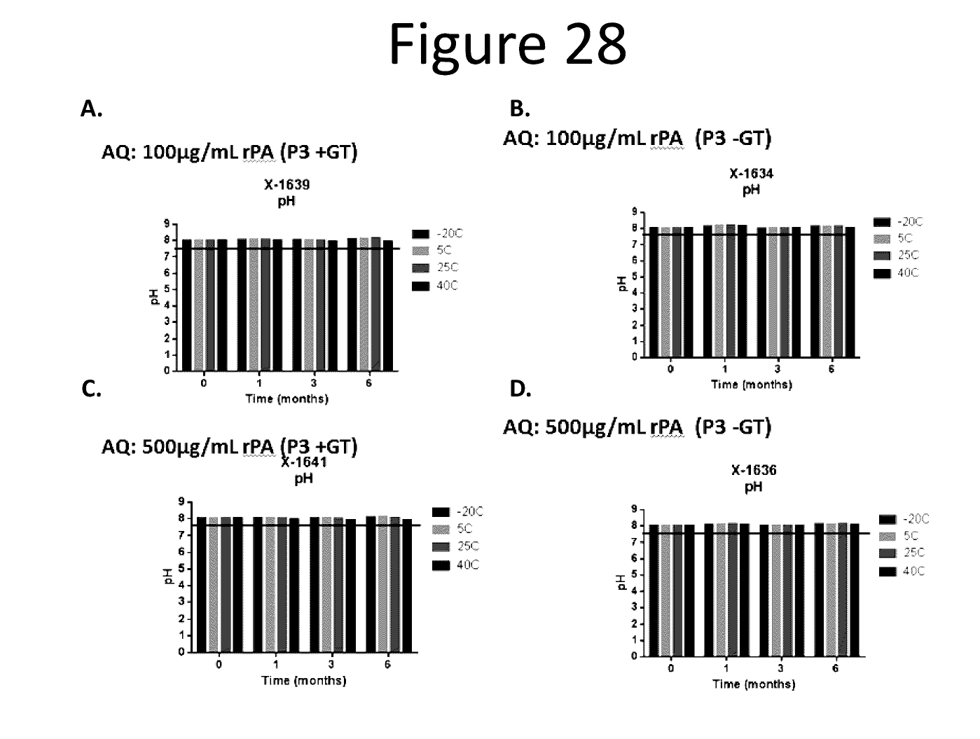

The pH was measured using a standard pH meter with the appropriate probe that can be used for both TRIS and PBS buffer systems. The formulations shown in Tables 5-7 are the formulations for which pH was assessed over time while storing the formulations at various temperatures. These results are shown in FIGS. 26-28.

### Example 9

### Particle Size Analysis and Polydispersity Index (PdI)

The mean particle size (Z-average) and polydispersity index (PdI) were determined for all the tested rPA samples. The particle size and PdI of the sample was measured by dynamic light scattering using photon correlation spectroscopy with a Malvern Zetasizer Nano ZS90 (Malvern Instruments, Worcestershire, UK). All measurements were carried out at 25° C. with no dilution.

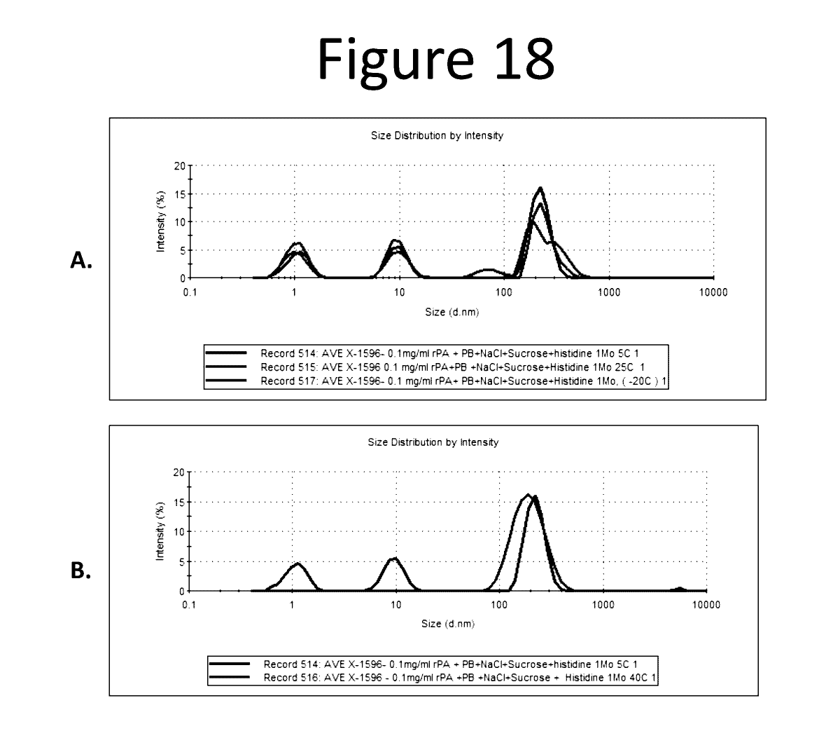

FIG. 18 shows the particle size profile of a 100 μg/mL rPA aqueous solution (Prototype 1: X-1596). It is apparent from the profile that the rPA particle size peak appears around 10 nm. The other two peaks are from the external phase buffer. FIG. 18A shows the solution at various one month stability temperatures of −20° C., 5° C., and 25° C. The rPA peak is retained. However, in FIG. 18B the rPA peak disappears at the 40° C., indicative of instability of the rPA at this temperature and time point.

### Example 10

### Label Claim of rPA by RP-HPLC or SEC-HPLC Test Method

The percent label claim (recovery) of rPA was determined using RP-HPLC and SEC-HPLC. Tables 10 and 11 describe the parameters of the each method.

TABLE 10
Size Exclusion HPLC Parameters for rPA determination.

| Parameter | Setting |
|---|---|
| Separation Mode | SEC |
| Stationary Phase | Tosoh Bioscience TSK-GEL G3000SWxL, |
| | 7.8 mm, 10 × 300 mm, L |
| Column Temperature | 25° C. |
| Run Time | 30-45 minutes (range for development purposes) |
| Flow Rate | 0.5 mL/min |
| Gradient/Isocratic | Isocratic |
| Mobile Phase | 0.1M Sodium Phosphate, 0.1M Sodium |
| | Sulfate, pH 6.8 |
| Sample Temperature | 4° C. |
| Injection Volume | 10 μL for formulations containing |
| | 500 μg/mL rPA |
| | 50 μL for formulations containing |
| | 100 μg/mL rPA |
| Detector Wavelength | 220 nm |
| Retention Time | 19.7 minutes with a guard column |
| | 17.7 minutes without a guard column |

TABLE 11
RP-HPLC HPLC Chromatographic Conditions
for rPA determination.

| | |
|---|---|
| Column: | ACE 5 Phenyl-300, 100 × 4.0 mm id, |
| | ACE Part Number: ACE-225-1004 |
| Elution Type | Gradient |
| Flow Rate | 0.5 mL/minute |
| Column Temperature: | 45° C. |
| Buffer A | 0.05% Trifluoroacetic Acid (TFA) in Water |
| Buffer B | 0.04% Trifluoroacetic Acid (TFA) in Acetonitrile |
| Auto Sampler | 4° C. |
| Temperature: | |
| Injector Volume: | 10 μL or |
| rPA Strengths: | For 10 μL: 2.5 ppm, 5 ppm, 10 ppm, 20 ppm, |
| | 25 ppm, 50 ppm, 100 ppm, 150 ppm, 200 ppm |
| | For 50 μL: 2.5 ppm, 5 ppm, 10 ppm, 20 ppm, |
| | 25 ppm, 50 ppm, 100 ppm |
| Detector Wavelength: | 214 nm |
| Run Time: | 37.5 minutes |
| Retention Time: | 12.5 minutes |

Stability studies of aqueous rPA formulations without stabilizing excipients were initiated. The compositions of the formulations are presented in Table 12.

TABLE 12
rPA Formulation in 10 mM Phosphate
Buffer Solution with 100 mM NaCl.

| | | Pre-Prototype Compositions | | | |
|---|---|---|---|---|---|
| | | rPA | % | Buffer | NaCl |
| Lot # | Type | (μg/mL) | NE | System | (mM) |
| X-1668 | rPA aqueous | 100 | 0 | 10 mM PBS | 100 |
| X-1670 | rPA aqueous | 500 | 0 | 10 mM PBS | 100 |

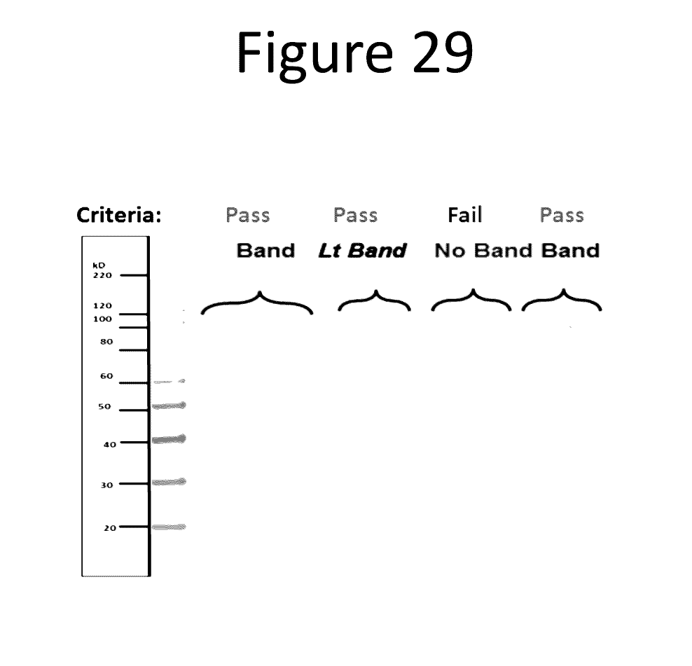

The rPA concentrations tested for stability, bracket at 100 μg rPA/mL and 500 μg rPA/mL. The rPA formulations were stored at −20° C., 5° C., and 25° C., and the stability of the rPA formulation was assessed at 1, 3, and 6 months. Formulations were also placed at 40° C. and analyzed at 1, 3, and 6 months. The rPA stability assays included: physical appearance, pH, particle size, and qualitative Western Blots for rPA, and % rPA label claim. % rPA label claim was determined by RP-HPLC and SEC-HPLC. The Western Blots for this set of formulations are not shown, but the acceptance criteria for the qualitative Western Blot method are shown in FIG. 29. If there is an 83 kDA band present or a light band, then it was considered to pass, as shown in lanes 1-5 after the molecular weight ladder. If no band is present, as shown in lanes 7 and 8, that was considered a failure.

The purpose of this experiment was to test rPA in a 10 mM phosphate buffered system with 100 mM NaCl without any stabilizing excipients.

Table 13 shows the stability data of a low dose (100 μg/mL) rPA, aqueous formulation (X-1668) in a phosphate buffer without any stabilizing excipients. It was stable for 3 months at 5° C. and 25° C. However, the high dose (500 μg/mL rPA) rPA aqueous formulation (X-1670) shown in Table 14 showed to be less stable. X-1670 was stable at 3 months at 5° C., but failed at 25° C.

This data indicates that stabilizing excipients are needed to help improve the stability of rPA present in an aqueous formulation at higher temperature for a longer duration.

TABLE 13
Overall Summary of 100 μg/mL rPA in 10 mM Phosphate Buffer
with 100 mM NaCl.

| | | | | | | Western | rPA—HPLC |
|---|---|---|---|---|---|---|---|
| | | | | | | Blot | RP |
| Time | Storage | Physical | pH | Particle Size | | (−83 kD | (SEC) |
| Point | Condition | Appearance | (±0.5) | (nm) | PdI | Band) | (>80%) |
| 0 | Initial | Pass | 7.49 | 8.26 | — | Band | 98 (98) |
| 1 month | 5° C. | Pass | 7.40 | 8.6 | — | Band | 87 (90) |
| | 25° C./60% RH | Pass | 7.42 | 8.0 | — | Band | 95 (0) |
| | 40° C./75% RH | Pass | 7.52 | 0 | — | Lt Band | 29 (0) |
| 3 month | 5° C. | Pass | 7.42 | 8.7 | — | Band | 100 (100) |
| | 25° C./60% RH | Pass | 7.52 | 7.5 | — | Band | 96 (93) |
| | 40° C./75% RH | Pass | 7.82 | 0 | — | No Band | 4 (0) |
| 6 months | 5° C. | Pass | 7.38 | 9.78 | — | Band | 86(89) |
| | 25° C./60% RH | Pass | 7.37 | 7.94 | — | No Band | 74(71) |
| | 40° C./75% RH | Pass | 7.55 | ND | — | ND | 4/0 |

TABLE 14
Overall Summary of 500 μg/mL rPA in 10 mM Phosphate Buffer with
100 mM NaCl

| | | | | | | rPA— |
|---|---|---|---|---|---|---|
| | | | | | Western | HPLC |
| | | | Particle | | Blot | RP |
| Time | Storage | Physical | Size | | (−83 kD | (SEC) |
| Point | Condition | Appearance | (nm) | PdI | Band) | (>80%) |
| 0 | Initial | Pass | 8.45 | — | Band | 95 (98) |
| 1 | 5° C. | Pass | 8.3 | — | Band | 97 (100) |
| month | 25° C./60% RH | Pass | 7.4 | — | Band | 95 (97) |
| | 40° C./75% RH | Fail | 0 | — | Lt Band | 5 (3) |
| 3 | 5° C. | Pass | 8.2 | — | Band | 101 (105) |
| month | 25° C./60% RH | Pass | 0 | — | No band | 0 (0) |
| | 40° C./75% RH | Fail | 0 | — | No Band | 0 (0) |
| 6 | 5° C. | Pass | 8.2 | — | No Band | 93(92) |
| months | 25° C./60% RH | Pass | 0 | — | No Band | 0/0 |
| | 40° C./75% RH | Fail | 0 | — | No Band | 0/0 |

Stability studies of various rPA aqueous formulations were initiated on the formulations shown in Table 5. The previous screening stability studies helped to guide formulation development and final formulation selection. The first prototype series was two sets of formulations comprising either phosphate or TRIS buffer. The test methods and acceptance criteria for the formulations placed on stability are shown above. The rPA concentrations shown for stability, bracket at 100 μg rPA/mL and 500 μg rPA/mL. The rPA formulations were stored at −20° C., 5° C. and 25° C. and stability was assessed at 1, 3, 6, 9, and 12 months. Formulations were also placed at 40° C. and were analyzed at 1, 3, and 6 months. The stability assays include: physical appearance, pH, particle size, and qualitative Western Blots. At later time points, rPA recovery was determined by RP-HPLC and SEC-HPLC.

The purpose of this set was to select the best buffer for between PBS and TRIS. It was evident that the TRIS System was superior to PBS in stabilization of rPA in formulations. At low dose 100 μg/mL rPA, the PBS system showed rPA stability at 3 months at 5° C. However, at high dose 500 μg/mL rPA, the PBS system only had 6 months at 5° C., while the TRIS system provided stability of rPA for 12 months at 5° C. for the high dose.

### Example 11

### Stability Data for rPA Prototype 2 Formulations (TRIS with 5% or 15% Trehalose)

The second prototype was two sets of rPA formulation comprising either 5% or 15% trehalose in a TRIS buffered system as shown in Table 6. The test methods and acceptance criteria for the formulations placed on stability are shown in Table 8. The rPA concentrations shown for stability bracket at 100 μg rPA/mL and 500 μg rPA/mL. The rPA formulations were stored at −20° C., 5° C., and 25° C. and stability was assessed at 1, 3, 6 and 9 months. Formulations were also placed at 40° C. and analyzed at 1, 3 and 6 months. The stability assays include: physical appearance, pH, particle size, and qualitative Western Blots. rPA recovery was determined by RP-HPLC and SEC-HPLC.

The purpose of this set was to select the best concentration of trehalose to be incorporated in a TRIS buffered system. rPA aqueous systems showed equivalent stability profiles except for the low dose rPA aqueous system. The low dose (100 μg/mL rPA aqueous system) was stable for 6 months at 5° C., while all the other systems were stable at 9 months at 5° C. The pH was stable for all the temperatures, except for 40° C. for 6 months. This is an improvement in the pH stability profile as compared to the rPA Prototype 1 formulations. The rPA potency by RP-HPLC/SEC-HPLC best shows the stability differentiation of the formulations. The potency of rPA in the rPA aqueous systems at the 25° C. condition from 1 to 6 months ranges from 40-85%.

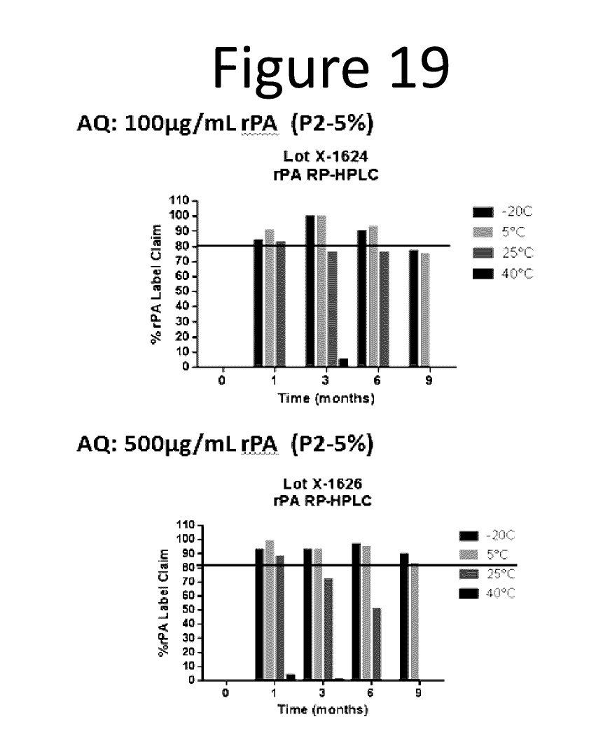

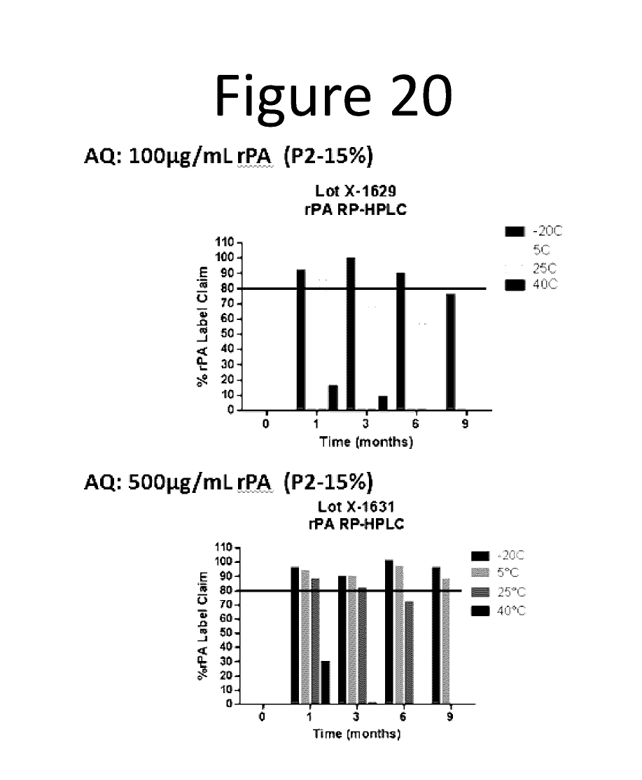

With respect to the level of trehalose, the benefit of increasing the trehalose from 5% to 15% is clearly demonstrated in FIGS. 19-20.

This increase in levels of stable rPA indicates that the additional trehalose helps protect rPA at high temperatures over a longer duration of time as compared to 5% trehalose.

### Example 12

### Stability Data of Prototype 3 (TRIS Buffered System with/without Glutathione) Formulations

The third rPA prototype was two sets of rPA formulations with or without 16 mM Glutathione in a TRIS buffered system as shown in Table 7. The rPA concentrations are bracketed at 100 μg rPA/mL and 500 μg rPA/mL. The formulations were stored at −20° C., 5° C., and 25° C., and stability was assessed at 1, 3 and 6 months. Formulations were also placed at 40° C. and analyzed at 1, 3 and 6 months. The stability assays include: physical appearance pH, particle size, and qualitative Western Blots. The Western blots were performed using the harmonized Western Blot method for rPA and the Novus rabbit polyclonal whole sera antibody as the primary antibody. The rPA recovery was determined by RP-HPLC and SEC-HPLC.

The purpose of this set of rPA prototypes was to understand the contribution of glutathione and histidine when incorporated in a TRIS buffered system.

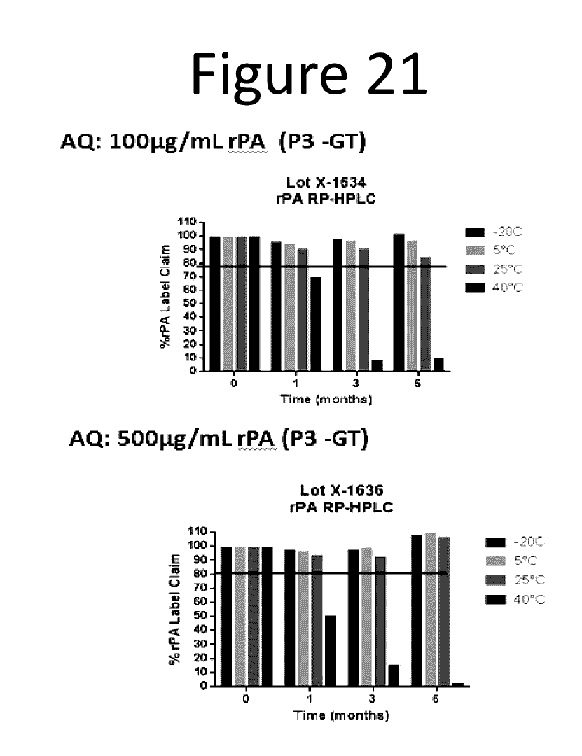

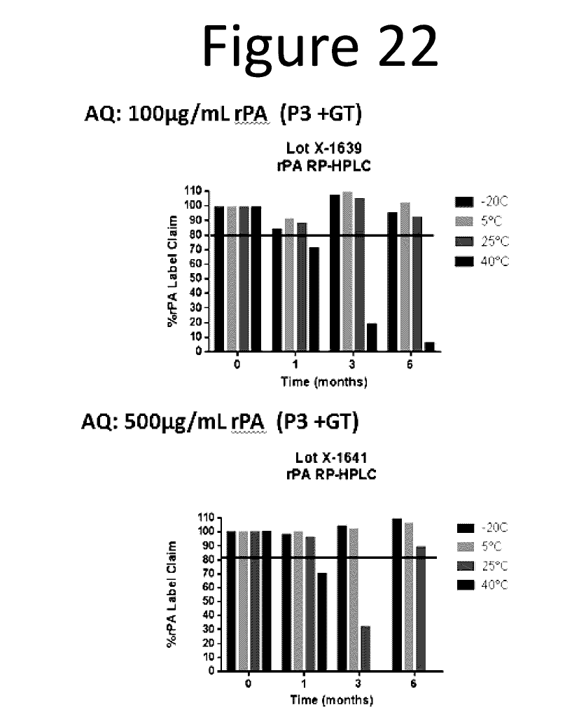

FIGS. 21 and 22 show the rPA recovery over time and temperatures for the rPA aqueous systems. The rPA recovery in the rPA aqueous systems at 25° C. was above 80% for every formulation tested. This is an improvement over the rPA aqueous systems from Prototype 2 which ranged from 40% to 80%.

With respect to the addition of glutathione, there does not appear large benefit of this excipient for rPA stability. When rPA potency is compared with and without glutathione, there is little effect on rPA recovery when measured using RP-HPLC.

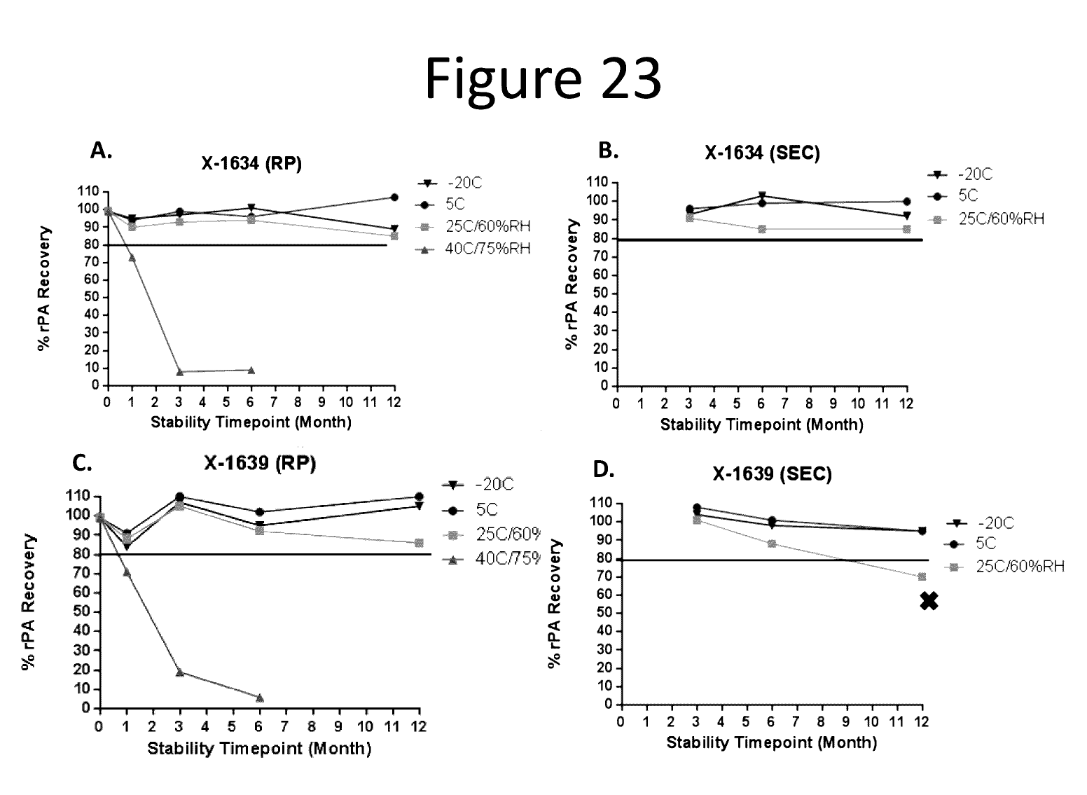

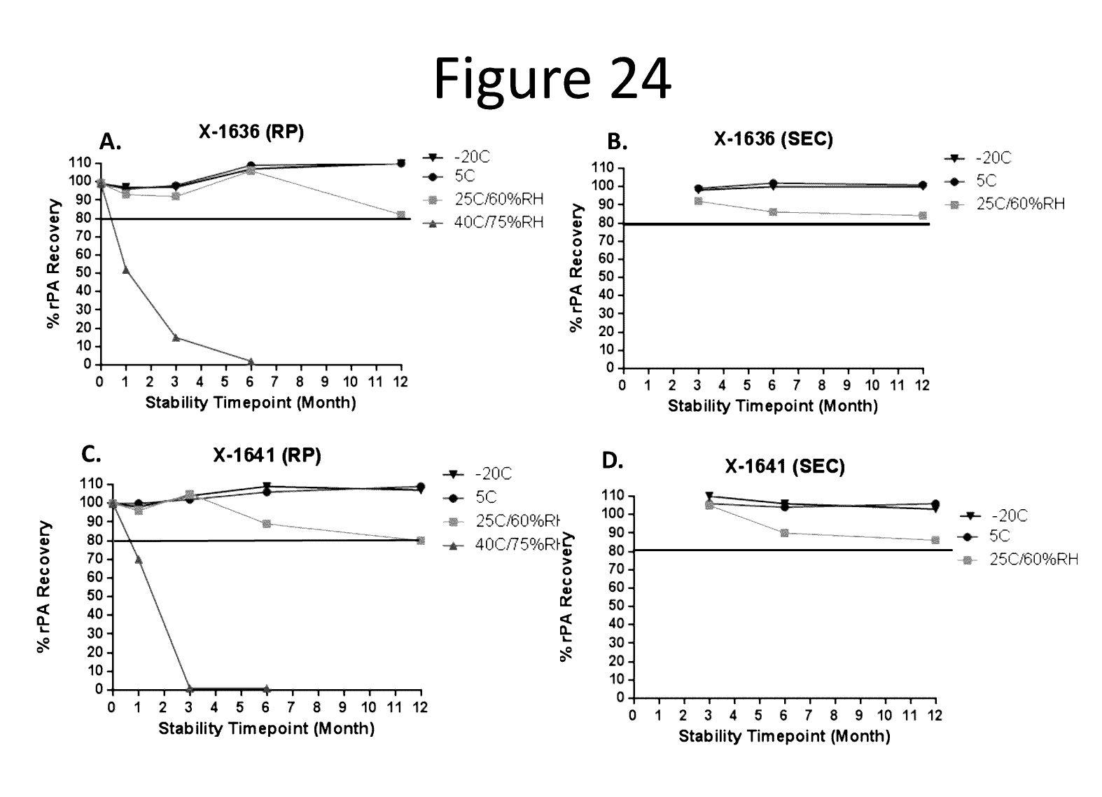

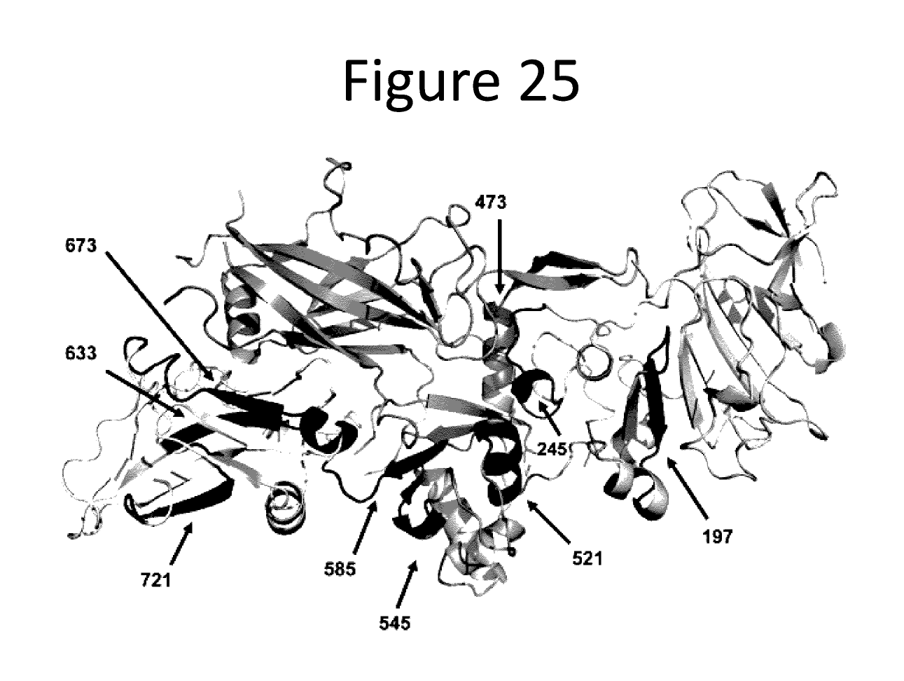

FIGS. 23 and 24 show a comparison of the RP and SE-HPLC methods. Here the lower concentration rPA formulation is less stable with the incorporation of glutathione while the high concentration formulation is stable as determined by SE-HPLC.

The low dose rPA aqueous solutions without glutathione has 12 months of rPA stability at 25° C. as measured by % rPA recovered with RP and SEC HPLC. When glutathione is incorporated, that stability is 12 months at 25° C. by RP-HPLC, but 12 months at 5° C. with SE-HPLC (see FIG. 23).

The high dose rPA aqueous solutions without glutathione have 12 months of rPA stability at 25° C. as measured by % rPA recovered with RP and SEC HPLC. When glutathione is incorporated, that stability is also 12 months at 25° C. by both methods RP-HPLC and SE-HPLC (see FIG. 24).

The above examples are given to illustrate the present invention. It should be understood, however, that the spirit and scope of the invention is not to be limited to the specific conditions or details described in these examples. All publicly available documents referenced herein, including but not limited to U.S. patents, are specifically incorporated by reference.

What is claimed:

1. A method of stabilizing anthrax recombinant protective antigen (rPA) in a composition, comprising formulating the rPA protein in a stabilizing system, wherein the stabilizing system comprises:
(a) TRIS buffer;
(b) at least one salt;
(c) at least one sugar; and
(d) at least one amino acid.

2. The method of claim 1, wherein:
(a) the TRIS buffer is in a concentration of about 5 to about 100 mM;
(b) the TRIS buffer is in a concentration of about 10 mM; or
(b) the TRIS buffer is in a concentration of about 80 mM.

3. The method of claim 1, wherein the salt is sodium chloride or calcium chloride.

4. The method of claim 3, wherein the sodium chloride is in a concentration of about 50-about 150 mM.

5. The method of claim 3, wherein the calcium chloride is in a concentration of about 50-about 150 mM.

6. The method of claim 1, wherein the sugar is trehalose.

7. The method of claim 6, wherein the trehalose is in a concentration of about 5-about 15%.

8. The method of claim 1, wherein the amino acid is histidine.

9. The method of claim 8, wherein:
(a) the histidine is in a concentration of about 20 to about 70 mM; or
(b) the histidine is in a concentration of about 60 mM.

10. A stabilized recombinant protective antigen (rPA) composition, comprising:
(a) anthrax recombinant protective antigen (rPA); and
(b) a stabilizing system comprising:
(i) TRIS buffer;
(ii) at least one salt;
(iii) at least one sugar; and
(iv) at least one amino acid.

11. The composition of claim 10, wherein:
(a) the TRIS buffer is in a concentration of about 5 to about 100 mM;
(b) the TRIS buffer is in a concentration of about 10 mM; or
(c) the TRIS buffer is in a concentration of about 80 mM.

12. The composition of claim 10, wherein the salt is sodium chloride or calcium chloride.

13. The composition of claim 12, wherein the sodium chloride is in a concentration of about 50 to about 150 mM.

14. The composition of claim 12, wherein the calcium chloride is in a concentration of about 50-about 150 mM.

15. The composition of claim 10, wherein the sugar is trehalose.

16. The composition of claim 15, wherein the trehalose is in a concentration of about 5 to about 15%.

17. The composition of claim 10, wherein the amino acid is histidine.

18. The composition of claim 17, wherein:
(a) the histidine is in a concentration of about 20 to about 70 mM; or
(b) wherein the histidine is in a concentration of about 60 mM.

19. The composition of claim 10, wherein the composition is formulated into a pharmaceutical composition.

20. The composition of claim 10, wherein the composition is formulated into a vaccine.
